(19) United States
(12) Patent Application Publication
(10) Pub. No.: US 2022/0079527 A1
(43) Pub. Date: Mar. 17, 2022

(54) SYSTEMS AND METHOD PROVIDING A UNIFIED FRAMEWORK FOR DE-NOISING NEURAL SIGNALS

(71) Applicant: University of Houston System, Houston, TX (US)

(72) Inventors: Jose Luis Contreras-Vidal, Houston, TX (US); Atilla Kilicarslan, Houston, TX (US)

(73) Assignee: University of Houston System, Houston, TX (US)

(21) Appl. No.: 17/422,248

(22) PCT Filed: Jan. 13, 2020
(86) PCT No.: PCT/US20/13285

§ 371 Date: Jul. 12, 2021

Related U.S. Application Data
(60) Provisional application No. 62/801,242, filed on Feb. 5, 2019.
(60) Provisional application No. 62/791,564, filed on Jan. 11, 2019.

(51) Int. Cl. A61B 5/00; A61B 5/369; A61B 5/398; A61B 5/11; A61B 5/31; G16H 40/40

(52) U.S. Cl. CPC A61B 5/721; A61B 5/369; A61B 5/398; A61B 2562/0219; A61B 5/31; G16H 40/40; A61B 5/1102

(57) ABSTRACT

A method removing artifacts from neural signals comprises receiving electroencephalography (EEG) data from an EEG system and providing the EEG data to a unified artifact removal framework for cleaning artifacts. The EEG data is provided to a first cleaning framework utilizing the first reference to clean first artifacts from the EEG data, and the outputting the EEG data from the first cleaning framework to a second cleaning framework. The second cleaning framework may operate in a similar manner utilizing second reference to clean second artifacts from the EEG data. This general process may be repeated as desired to clean various artifacts from EEG data, when a suitable reference for the artifacts to be removed is utilized. The frameworks utilize H∞ method or filtering involving a H∞ adapting rule to properly weigh the reference, and combining the subsequent output with incoming EEG data results in the desired removal of artifacts.

### RELATED APPLICATIONS

This application claims the benefit of U.S. Provisional Patent Application Nos. 62/791,564 filed on Jan. 11, 2019 and 62/801,242 filed on Feb. 5, 2019, which are incorporated herein by reference.

### STATEMENT REGARDING FEDERALLY SPONSORED RESEARCH

This invention was made with government support under Grant Nos. R01NS075889 from the National Institute of Neurological Disorders and Stroke (NINDS) and 1827769 from the National Science Foundation (NSF). The government has certain rights in the invention.

### FIELD OF THE INVENTION

This invention relates to a framework for de-noising neural signals. More particularly, to a unified artifact removal framework for removing various artifacts.

### BACKGROUND OF INVENTION

Electroencephalography (EEG) is an electrophysiological monitoring method to record electrical activity of the brain. It is typically noninvasive, with the electrodes placed along the scalp, although invasive electrodes are sometimes used, as in electrocorticography. EEG measures voltage fluctuations resulting from ionic current within the neurons of the brain. EEG has several applications, including nonlimiting examples, such as utilizing EEG to control exoskeletons or prosthetics (e.g. U.S. Pat. Nos. 9,468,541, 10,092,205, WO 2017/218661).

The brain signals from an EEG are prone to artifacts, such as motion or ocular artifacts, in many different types of clinical and technological applications. EEG is perhaps the most widely used non-invasive brain wave measurement system for various purposes. However, all EEG systems are prone to artifacts, which make the analysis of true brain signals very difficult. This motion artifact contamination affects results, especially when mobile applications such as walking or free body movement are considered, as motion artifacts greatly impact the EEG signals. There is currently no method of identifying and removing (i.e., denoising) these artifacts.

Accurate implementation of real-time neural interfaces requires handling major physiological and non-physiological artifacts that are associated with the measurement modalities. As EEG measurements are prone to excessive motion artifacts and other types of artifacts that contaminate the EEG recordings, it is very difficult to provide accurate implementation. Although the magnitude of such artifacts heavily depends on the task and the setup, complete minimization or isolation of such artifacts is extremely difficult.

A clear consensus among researchers is that the motion artifacts are highly dynamic in nature, and are affected by the setup and movement dynamics. It is also highly variable among subjects, within the same session and with respect to the scalp spatial location of EEG sensors of the same subjects. While research has been conducted on such artifacts, there is still no consensus on how to handle these artifacts when they manifest themselves, how to characterize their conditional variabilities and perhaps even less examined, how to remove/suppress them in real-time.

### SUMMARY OF INVENTION

In one embodiment, a method removing artifacts from neural signals comprises receiving electroencephalography (EEG) data from an EEG system and providing the EEG data to a unified artifact removal framework for cleaning artifacts. The EEG data is provided to a first cleaning framework utilizing the first reference to clean first artifacts from the EEG data, and the outputting the EEG data from the first cleaning framework to a second cleaning framework. The second cleaning framework may operate in a similar manner utilizing second reference to clean second artifacts from the EEG data. This general process may be repeated as desired to clean various artifacts from EEG data, when a suitable reference for the artifacts to be removed is utilized. The frameworks utilize H^{∞} method or filtering involving a H^{∞} adapting rule to properly weigh the reference, and combining the subsequent output with incoming EEG data results in the desired removal of artifacts.

In yet another embodiment, a method removing artifacts from neural signals may be similar to the methods above. The first cleaning framework may be an ocular cleaning framework, and the second cleaning framework may be a motion artifact cleaning framework. Further, the unified artifact removal framework may comprise a BCG artifact removal framework, tACS artifact removal framework, or some combination of the various frameworks.

A system for removing artifacts from neural signal may provide an electroencephalography (EEG) system comprising a plurality of electrodes that gather EEG data. A unified artifact removal framework for cleaning artifacts from the EEG data may also be provided. The unified artifact removal framework may include one or more artifact removal frameworks, each providing a H^{∞} module receiving a reference utilized to clean artifacts from the EEG data, and a combiner combining an output of the Hoc module with the EEG data to clean the artifacts from the EEG data. Fully clean EEG data is output from the unified artifact removal framework. The system may also a processor controlling various operations of the unified artifact removal framework.

The foregoing has outlined rather broadly various features of the present disclosure in order that the detailed description that follows may be better understood. Additional features and advantages of the disclosure will be described hereinafter.

### BRIEF DESCRIPTION OF THE DRAWINGS

For a more complete understanding of the present disclosure, and the advantages thereof, reference is now made to the following descriptions to be taken in conjunction with the accompanying drawings describing specific embodiments of the disclosure, wherein:

FIG. 1 shows a nonlimiting embodiment of a system suitable for the framework for de-noising neural signals;

FIG. 2 shows a nonlimiting example of a workspace of a movement data collection setup utilized for experimentation;

FIG. 3 shows an embodiment of an EEG system coupled to module and prosthetic or exoskeleton;

FIG. 4 shows an adaptive filter framework or methodology;

FIG. 5 shows H^{∞} and ICA decomposition weights for cleaning EEG data from all scalp electrodes;

FIGS. 6A-6C show time course of real-time filtering results for select channels;

FIGS. 7A-7G show power spectral densities of VEOG, HEOG, raw EEG, ICA, ASR and H^{∞} filtered data;

FIGS. 8A-8C provide panels of scalp topographical plots of EEG contamination, time traces for select electrodes, and components of eye blinks on select electrode locations;

FIGS. 9A-9D show group correlations for all subjects and marked electrodes for different walking speeds;

FIGS. 10A-10D show group coherence for all subjects and marked electrodes for different walking speeds;

FIG. 11 shows the grand average PSD of EEG data from all marked electrodes for different walking speeds;

FIGS. 12A-12F shows topographical plots for various electrodes and before/after filtering of motion artifacts, idetified artifacts and vertical head accelerations, and power spectra;

FIGS. 13A-13F show topographical plots and raw/clean EEG signals, identified artifacts and vertical head accelerations, and power spectra;

FIG. 14 shows power spectra of the raw and clean EEG signals for select electrodes at different walking speeds;

FIG. 15 shows time-frequency plot of a segment of EEG data (C5 electrode) at 2 mph walking speed before and after motion artifact cleaning;

FIG. 16 shows raw and cleaned ERSP of the C5 electrode for all walking speeds for the same subject;

FIGS. 17A-17B show simultaneous tACS/EEG stimulation artifact cleaning for 10 Hz discontinuous stimulation;

FIGS. 18A-18B show simultaneous tACS/EEG stimulation artifact cleaning for 6 Hz discontinuous stimulation;

FIGS. 19A-19B show simultaneous tACS/EEG stimulation artifact cleaning for 20 Hz discontinuous stimulation;

FIG. 20 shows the before and after BCG cleaning of data for select channels; and

FIGS. 21A-21B show a segment of raw and clean EEG data for the intermittent tACS stimulation paradigm, and the corresponding PSDs before and after cleaning.

### DETAILED DESCRIPTION

Refer now to the drawings wherein depicted elements are not necessarily shown to scale and wherein like or similar elements are designated by the same reference numeral through the several views.

Referring to the drawings in general, it will be understood that the illustrations are for the purpose of describing particular implementations of the disclosure and are not intended to be limiting thereto. While most of the terms used herein will be recognizable to those of ordinary skill in the art, it should be understood that when not explicitly defined, terms should be interpreted as adopting a meaning presently accepted by those of ordinary skill in the art.

It is to be understood that both the foregoing general description and the following detailed description are exemplary and explanatory only, and are not restrictive of the invention, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

An inertial measurement unit (IMU) is an electronic device that measures and reports a body's specific force, angular rate, and sometimes the orientation of the body, using a combination of accelerometers, gyroscopes, and sometimes magnetometers.

Unified De-Noising Systems

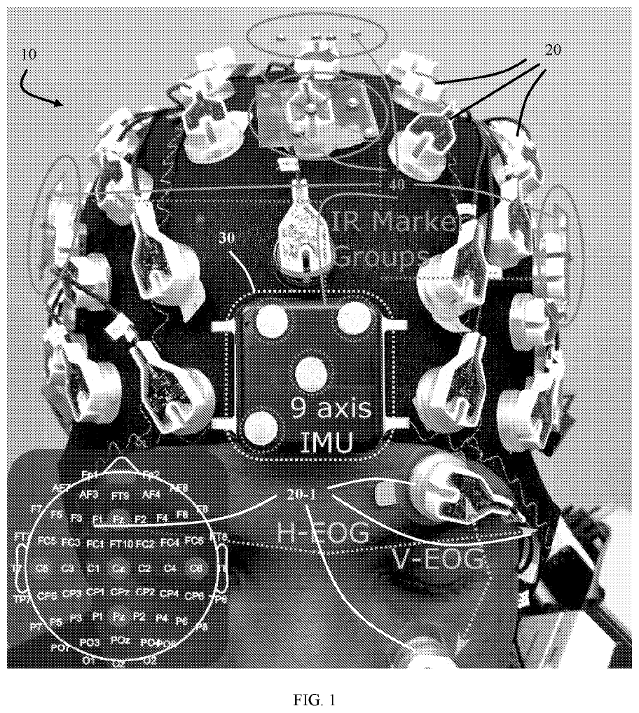

FIG. 1 shows a nonlimiting embodiment of a system suitable for the framework for de-noising neural signals. Any suitable electroencephalography (EEG) system 10 may be utilized. A nonlimiting example of the EEG system 10 may be a cap providing a plurality of electrodes 20 for gathering EEG data or signal(s). As shown, electrodes of the EEG system may generally be placed at known locations. In some embodiments, a subset of EEG channels 20-1 may be assigned for placement at select locations. As a nonlimiting example, the subset of EEG channels may be assigned as bipolar horizontal and vertical EOG (electrooculography) channels 20-1. As shown, a subset of four electrodes 20-1 may be positioned on left and right temples (H-EOG) and above and below an eye (V-EOG), which may aid monitoring horizontal and vertical ocular artifacts respectively. In some embodiments, motion artifact removal may be desired, thereby necessitating placement of a subset of electrodes at desired locations. In some embodiments, ballistocaridiography (BCG) artifact removal may be desired, thereby necessitating placement of a subset of electrodes at desired locations. In some embodiments, transcranial Alternative Current Stimulation (tACS) artifact removal may be desired, thereby necessitating placement of a subset of electrodes at desired locations. As a nonlimiting, 5 channels (e.g. T7, CP2, CP5, FC3, CP3) may be assigned. In some embodiments, peripheral electrodes FT9 and FT10 may be moved to FPz and FCz locations for a denser scalp coverage. In some embodiments, Reference and Ground electrodes may be moved to the ears. An external layer on EEG electrodes was not used (e.g., a medical mesh) in experimentation, as artifacts were desired in experimentation to verify viability—however, an external mesh may be desirable in other embodiments as it may dramatically reduce the motion of the electrodes and cable sway. In some embodiments, at least one IMU sensor 30 may be provided, which may be place at a desire location on a subject, such as the forehead, left & right foot, or the like. As a nonlimiting example, a 9 axis IMU sensor is shown placed at the subject's forehead, and/or IMU sensors may be optionally placed on the left and right foot. In some embodiments, markers 40 may be optionally placed on select electrodes (e.g. Fz, Cz, Pz, C5, C6, forehead IMU sensor).

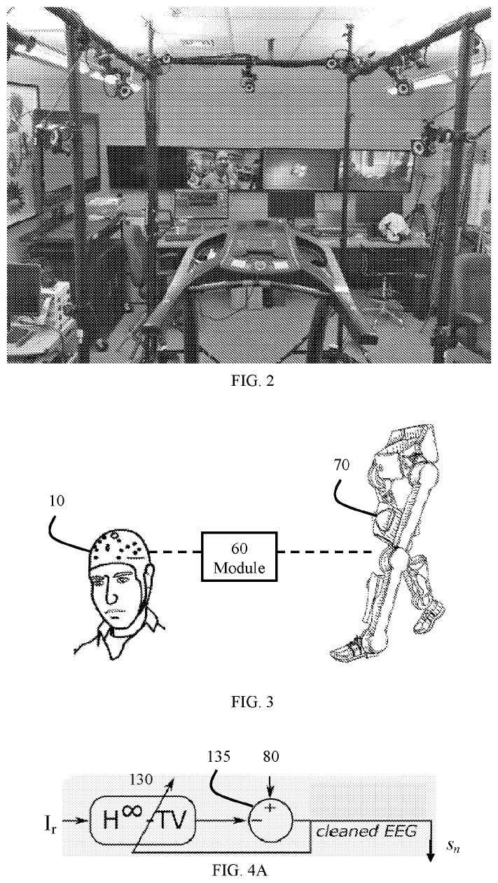

FIG. 2 shows a nonlimiting example of a workspace of the movement data collection setup utilized for experimentation, which is not needed in during normal operation, to verify viability of these improved systems and methods. A treadmill was surrounded by a motion tracking system, for which the approximate capture area was limited to the head of each subject. Cameras were calibrated before each session. Subjects were asked to walk on a treadmill while data was collected to verify viability.

In some embodiments, the system may be utilized for operating a prosthetic or exoskeleton. EEG data may be utilized to operate a prosthetic or exoskeleton via a brain-to-machine interface or BMI (e.g. U.S. Pat. No. 10,092,205 and WO 2017/218661). FIG. 3 shows an embodiment of the EEG system 10 coupled to module 60. Module 60 may comprise memory for storing data and/or firmware, software, and the like, as well a processor, CPU, microprocessor, or the like. Generally, the processor/CPU may utilize the software/firmware to control various operations desired. In some embodiments, the module 60 may provide a BMI for operating a prosthetic or exoskeleton 70. As a nonlimiting example, module 60 may provide motor intent decoding software implemented by a processor to analyze EEG data. The BMI 60 is responsible for decoding brain activity data, or raw EEG data from system 10, to determine motor intent, and controlling the prosthetic or exoskeleton 70 accordingly. To properly decode motor intent, it may be desirable in some embodiments to remove artifacts, which may impede decoding, from raw EEG signals from the EEG system 10, such as by utilizing a unified artifact removal framework discussed herein. In some embodiments, the module 60 may also provide the unified artifact removal framework discussed further herein. As a nonlimiting example, memory of the module 60 may provide artifact removal software implemented by the processor to perform the framework or processes discussed further herein on raw EEG signal(s), thereby removing undesired artifacts to provide clean EEG data.

Unified Artifact Removal Framework

The unified artifact removal frameworks utilizes H^{∞} methods or filtering methods discussed herein formulated around the robustness properties due to exogenous effects and modeling errors, and provide a comprehensive framework for real-time applications. H^{∞} methods may be generally characterized as adaptive cleaning or filtering methods where artifacts or disturbances are cleaned from an input, and feedback is utilized to adaptively adjust filtering.

The systems and methods discussed herein expand on H^{∞} filtering methods utilized for the unified artifact removal framework, such as ocular, motion, ballistocardiographic (BCG), transcranial Alternative Current Stimulation (tACS), and/or other artifact removal. It is noted that successful implementation of unified artifact removal framework, characterized as adaptive to remove artifacts, requires a good reference source of the contaminants, disturbances, or artifacts. Any neural activity that might be superimposed on or combined with the negative of reference noise sources, would be filtered by an optimal framework. As a nonlimiting example, EEG electrodes (such as FP1, FP2, FT9, FT10) are expected to include some neural activity including artifacts, which are desirable to eliminate. In some embodiments, dedicated artifact tools (e.g. EOG or IMU) may be desirable to generate a reference signal for the framework and/or obtain the optimal filter performance. As discussed previously, the electrodes of an EEG gathering neural activity data may be coupled to a unified artifact removal framework. The framework may be provide by a memory for storing data, software, or the like; a processor for implementing the de-noise/artifact removal discussed further herein; and/or input/output port(s) for receiving sending data.

The H^{∞} filter or H^{∞} adaptation rule may utilize a number of key parameters to approach its optimal filter behavior. These parameters tell the filter the level of contamination to handle, and how fast to adapt to the contaminants. In a multi-channel recording paradigm, the level of contamination and contamination profile varies dramatically among electrodes. Therefore, keeping the estimator parameters the same for the entire scalp hinders the true overall performance of the robust filter. In some embodiments, a parameter selection method may be used for automatic identification of filter parameters. The framework may be used to augment various artifacts or EEG contaminants, such as ocular contamination, motion artifacts, BCG artifacts, tACS artifacts, local/global drifts, and/or signal amplitude biases from EEG sources in a real-time setting. The simultaneous filtering of all these unwanted effects is discussed in detail herein.

In the unified framework, successful filtering of artifacts requires a good identification of artifact projections over all scalp areas or the reference utilized. This gives us the opportunity to assess the level of contamination for different scalp locations and also their sample-by-sample change over time. This property may be used to comment on the level of contamination due to volume conduction and get a clear snapshot of it over all scalp areas.

The real-time usability and performance characteristics of a H^{∞} filter was investigated, which takes advantage of full characterization of ocular and motion contaminants in a sample-by-sample adaptation scheme. Coupled with the robustness characteristics of the H^{∞} formulation in general, this method is inherently less sensitive (or more robust) to dynamically changing characteristics of artifacts or contaminants. Importantly, this method does not rely on the definition of clear EEG segments, and does not require the acquisition of clean EEG segments, as in an ASR method. Furthermore, the method discussed may allow operators to selectively remove what they desire to remove from EEG data.

The non-stationary nature of the EEG, as well as the artifacts superimposed on them, makes the selection of cleaning methodology exceedingly important. The changes in the characteristics of the artifacts over short periods of time require an adaptation scheme that can effectively handle these dynamics. An adaptive robust framework or H^{∞} filter formulation with time-varying weigh assumption as an estimator is desirable to compensate for the artifact variability, and identify the projections of the artifacts per channel of EEG data in a sample-adaptive fashion. The unified framework may include different individual frameworks corresponding to different artifacts to be removed from EEG data. For example, an ocular artifact cleaning framework is based on the robust H^{∞} filter/framework with time-varying weight formulation. To accomplish a fast and effective cleaning, a single weight value, per reference input, may be utilized and the weights may be estimated with the H^{∞} formulation in some embodiments. The effectiveness of the robust-adaptive filter framework for the ocular artifacts, signal drift, and biases, as well as motion and other select artifacts, is shown by experimentation discussed further below. A linear implementation was found only partially effective, mainly limited to the major frequency of contamination, which is locked to the primary head movement frequency.

In some embodiments, the noise or artifact source (or reference that is input to the framework) is measured with dedicated sensors (e.g. EOG or IMU), and the components of these sources that are represented in the raw EEG data were identified for each sample of data. Although the dedicated sensors might seem like a disadvantage at first, it provides the means to be very specific in terms of the identified components and allows for the selective removal of artifacts from the EEG data. Methods that work only with the clean EEG and expected artifact statistics cannot guarantee the cleaning of outlier contamination and might even risk the removal of underlying neural components that are similar to the artifacts, statistically. In some embodiments, it may be desirable to pre-process the reference or artifact source provided to the framework. As a nonlimiting example, to accomplish a better estimation of the head movement projection to each individual EEG channel, a 2^{nd} order Volterra-series expansion of the reference inputs may be introduced, e.g. having also up to 3 samples time taps. Even though the implementation handled the major frequency contamination with great time and frequency domain suppression, the harmonics were not properly represented in experiments. Thus, a cascade filtering framework for all target frequencies may be utilized for some embodiments.

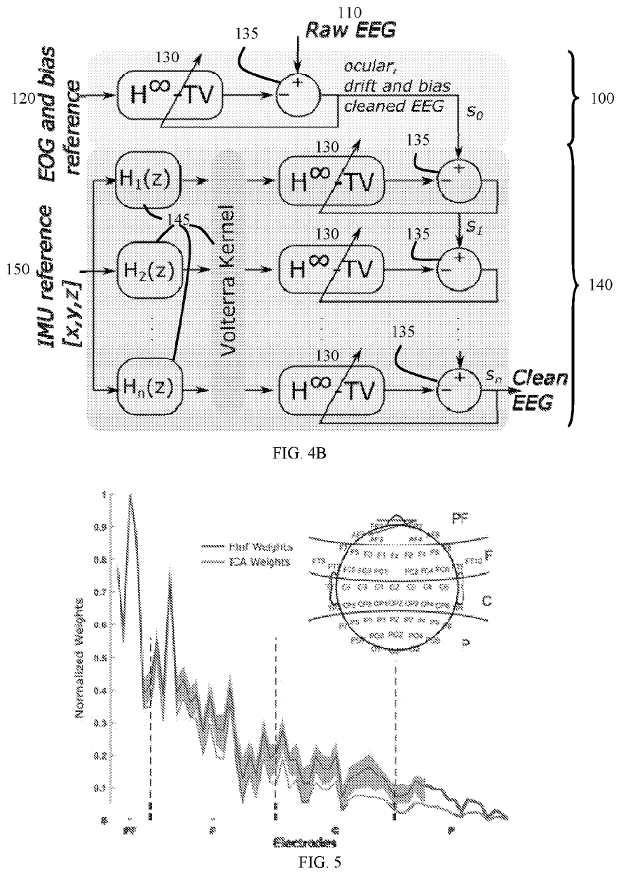

FIGS. 4A-4B shows a nonlimiting embodiment of a general structure of an adaptive filter framework or methodology for H^{∞} filtering, as well an overall structure for a unified artifact removal framework. The H^{∞} adaptation rule of the H^{∞} filtering method is common for ocular, motion, BCG, tACS, and/or other artifact removal tasks. As such, FIG. 4A shows a general framework suitable for removing various artifacts (e.g. ocular, motion, BCG, tACS, or the like). The framework receives an input or uncleaned EEG signal 80 (e.g. raw EEG, s₀, s₁, etc.), as well as a reference signal (or artifact reference) Iᵣ (e.g. EOG, IMU, etc.). In some cases, it may be optionally be desirable to perform pre-processing of the reference signal prior the signal being sent to H^{∞} module 130. In the H^{∞} module 130, a H^{∞} adaptation rule is performed on the reference signal Iᵣ (or pre-processed reference signal) to determine a desired weight for proper filtering, and a resulting output signal of H^{∞} module 130 is used to clean EEG signal 80. A combiner 135 is utilized to combine a negative of the resulting output signal after H^{∞} processing with the received EEG signal 80 to provide a clean signal or output sₙ, where n represents a cleaning stage of the framework. It shall be understood that the unified framework may comprise multiple individual framework(s), similar to the general framework of FIG. 4A, such as for providing frameworks for different artifact removal and/or providing multiple cascading stages.

FIG. 4B shows a nonlimiting example of an overall structure of a unified artifact removal framework for filtering multiple artifacts comprising an ocular cleaning framework 100 and a motion artifact or cascade filtering framework 140. As discussed previously, using artifact reference(s) (e.g. 120, 150), artifacts or disturbances can be determined utilizing a H^{∞} adaptation rule or module 130 (discussed further herein below), which may then be substracted from received EEG signal(s) (e.g. 110, s₀, s₁) to provide a clean signal (e.g. s₀, s₁ . . . sₙ). Notably, H^{∞} filtering 130 utilizes feedback from the output to adaptively adjust filtering, such as adjusting time varying weights. In some embodiments, it may be desirable to pre-process an incoming reference (e.g. 140). A 2^{nd} order Volterra series expansion was introduced per artifact reference source for the motion artifact problem. As a nonlimiting example, a 2^{nd} order Volterra series expansion may be applied to the reference signal or motion artifact references, such as the IMU references, to determine an unknown representations of the motion artifacts, prior to H^{∞} filtering. Further, in some embodiments, the unified and/or individual frameworks may be applied in multiple stages or in a cascading framework (e.g. 110, 140).

As a nonlimiting example, in some embodiments, raw EEG data or signal(s) 110 may be initially provided to an ocular cleaning framework 100. For example, the ocular artifacts data (e.g. EOG and bias reference) 120 may be processed by a H^{∞} module 130 utilizing a H^{∞} adaptation rule. H^{∞} module 130 generally apply a H^{∞} adaptation rule (discussed in detail below), which may also be referred to H^{∞} filtering or H°/TV (TV indicating the time-warying nature). The negative output of the module may be combined by combiner 140 with the incoming EEG data or signal 110 to provide clean EEG data or signal(s) s₀, particularly cleaned of ocular artifacts. In some embodiments, the output of the ocular cleaning framework may be provided to another artifact cleaning framework, such as a motion artifact framework or cascade filtering framework. For example, the EEG signal s₀ is provided to a cascade filtering framework 140 for removal of other artifacts, such as motion artifacts. The cascade filtering framework 140 receives IMU data or reference 150. As shown the cascading filtering framework 140 may provide multiple stages n, where input EEG progresses through multiple stages. EEG input generally refers to the input signal to be cleaned, which may be raw EEG data or EEG data previously cleaned of other artifacts in earlier stages. In some embodiments, each stage of the cascade filtering framework 140 may perform pre-processing of reference data, such as a 2^{nd} order Volterra-series expansions with Voterra module/kernel 145 on received reference signal(s) or IMU data 150, prior to further H^{∞} filtering. The corresponding output resulting from module/kernel 145 may then be processed by H^{∞} modules 130 (the output of which may be referred to a V-H^{∞}/TV) to clean the received EEG signal in a similar manner as discussed previously. For example, a received EEG signal s₀ is fed to a first stage, and IMU data 150 undergoes Volterra 145 and H^{∞} processing 130. The received EEG signal (e.g. s₀ in 1^{st} stage, s₁ in 2^{nd} stage, etc.) is combined with the negative output the processed IMU data 150, and an EEG output signal (s₁ . . . sₙ) that is clean of a portion of the artifacts in outputted. The cleaned EEG output from earlier stages (s₁ . . . sₙ) are feed to subsequent stages where similar processing is repeated. Once the initial EEG signal s₀ has progressed through the various stages of the cascade filtering framework 140, a clean EEG sₙ it output from the framework.

The H^{∞} processing and formulation is revisited in further detail below, as it applies to ocular artifacts, motion artifacts, or other artifact problems. Detailed discussion on the H^{∞} adaptation rule is also provided herein.

The H^{∞} Adaptation Rule

H^{∞} methods or filtering methods generally refers to any suitable methods for adaptively cleaning artifacts or disturbances from an input signal, such as EEG data. In some embodiments, such H^{∞} methods may involve a H^{∞} adaptation rule discussed further below. In some embodiments, H^{∞} methods may involve the H^{∞} adaptation rule with time-varying weight assumptions. In some embodiments, H^{∞} methods may also involve pre-processing, e.g. Volterra-series expansion of reference inputs, such as IMU data.

For comparison of techniques, FIG. 5 shows H^{∞} and ICA decomposition weights for cleaning EEG data from all scalp electrodes. Shaded region indicates the variability of H^{∞} weights and the solid line within the shaded region represent the mean weight. The other solid red represent the ICA weights per channel. The ocular artifact cleaning method discussed herein was used on the raw EEG data without any pre-processing. The drift and the bias of the data are also handled by the ocular framework and the resulting data after this process is identical to the raw data except for the artifactual time instances. FIG. 5 is chosen to emphasize the core advantage of using adaptive methods for de-noising the data discussed herein compared to the methods based on statistical metrics, such as ICA. This figure shows normalized values of the final ICA weights per channel that are used to clean ocular artifacts from the EEG data. The adaptive H^{∞} filter is a sample-adaptive formulation, therefore it allows for the adaptation to the change in contamination in time domain. The shaded region in the figure represents the minimum and maximum values of the weights that are adapted per sample of EEG data. The solid line in the shaded region is the average of all H^{∞} weights per channel. The similarities between the mean H^{∞} weights and the ICA weights suggest that the framework is capable of being selective of the ocular artifacts, very similar to the ICA method. The variation, on the other hand, is an added quality onto the weights, that addresses the time varying characteristics of the ocular artifacts. This improves the cleaning performance over ICA as discussed herein.

As previously mentioned, a number of adaptation schemes could be used for the unified de-noising system. Due to its robustness properties under modeling errors and unknown exogenous effects, a H^{∞} method was used for a sample by sample filter weight adaptation problem. The H^{∞} filtering formulation guarantees robustness such that, small modeling errors and exogenous noises does not cause large estimation errors. There are a number of different ways to formulate the H^{∞} filter weight estimation problem, out of concern of the convergence properties of the weights. In some embodiments, one can simply assume a fixed weight per EEG channel and request a linear or exponential convergence to the true weights. However, because of the inherent properties of EEG electrode measurements, such as changes in the percentage of artifact contamination on EEG channels over time due to external factors, convergence to a fixed weight problem poses limitations to the general robustness of such a framework. Nevertheless, a decent level of filtering could still be observed due to its sample-adaptive formulation. A safer and more robust way to adapt the weights would be the assumption of time varying weights per channel in some embodiments.

The H^{∞} adaptation rule with time-varying weight assumption is given as follows (B. Hassibi, T. Kailath, h^{∞} adaptive filtering, in: Acoustics, Speech, and Signal Processing, 1995. ICASSP-95, 1995 International Conference on, Vol. 2, IEEE, 1995, pp. 949-952):

$${\hat{w}}_{i + 1} = + \frac{{P}_{i} {r}_{i}}{1 + {r}_{i}^{T} {P}_{i} {r}_{i}} {y}_{i} for which , \\ {y}_{i} = {s}_{i} - {r}_{i}^{T} , and {P}_{i}^{- 1} = {\tilde{P}}_{i}^{- 1} - {γ}^{- 2} {r}_{i} {r}_{i}^{T} where$$

$${\tilde{P}}_{i + 1} = {[ {{\tilde{P}}_{l}}^{- 1} + ( 1 - {γ}^{- 2} ) {r}_{i} {r}_{i}^{T} ]}^{- 1} + qI$$

Here ŵᵢ represent the estimated weight vector of reference values, rᵢ the reference vector at sample i, sᵢ represents the raw EEG data, and \tilde{P}ᵢ is the noise covariance matrix initialized with \tilde{P}₀=μl where μ is a constant. T represents a matrix transpose operation, and I represents an identity matrix. The parameters γ and q play an important role on the behavior of the adaptive filter. γ determine the bound on the energy-to-energy gain from the disturbance to the estimation error, roughly determining the amount of disturbance that can be tolerated. For the time varying weight formulation, it should be selected as γ>1. This defines a sub-optimal filter as a trade-off for allowing the weights to vary. For the ocular artifact removal, γ≅1.15 is found to be effective, in general. The parameter q reflects the a priori information of how rapidly the weight will vary in time. Larger values covers for faster variations. For slow signals, q≅10⁻⁸ is usually a good start point. It should be noted that these parameters are application dependent.

In the above formulation, the weights are assumed to be time-varying with unknown variation dynamics, and the change δŵ is formulated as an unknown disturbance on the system. Hence, the above formulation is valid for the inequality γ²≤1+qr̅ where r̅ is the supremum of the measured disturbances. Although in a real-time setting there is no way to get an accurate estimate on the upper bound γ and supremum of the contaminant, one can readily measure a short set of data to determine better bounds, or simply use the training data of a BMI application. The deviation from optimal γ<1 formulation is a tradeoff of having a time-varying assumption on the weights. It is not implied that the linear or exponential convergence to a fixed weight vector formulation is not correct or suitable for EEG applications. However, especially for real-time applications that have uncontrollable circumstances, i.e. mobility of the subjects, changing electrode properties during long experiments and other factors that might affect the reflection of the artifact magnitudes on specific electrode measurements, makes it desirable to have a framework that can cover for those changes. In fact, these circumstances also justify the usage of robust filters over their H² optimal counterparts. Similar to the value γ, the parameter q also plays an important role on the filter behavior as it reflects the a priori information of how rapidly the weights vary with time.

Studies conducted examining the use of H^{∞} filter for artifact suppression indicate a single γ selected by trial and error and finding a value that works best by visually examining the filter behavior may be viable. The best pair of γ and q values was investigated for a given data set and their values per electrode. The reflection of ocular artifacts on different electrode locations is not expected to be the same due to volume conduction, nor can they be assumed as coupled systems, due to possibly different exogenous effects. Each electrode measurement is treated as a separate sub-system in some embodiments. Although in general, the weights of the H^{∞} filter will be adjusted accordingly in an adaptive setting, the performance of the overall filter can be improved upon by identifying the best pair of parameters per electrode. In experimentation, for this offline step of searching a pair of γ and q values, a constrained optimization problem (e.g. using Matlab® fmincon function) is formulated. In each iteration, the values γ and q may be updated using the cross correlation values of filtered EEG data to the raw EEG data (both windowed) as a cost function in some embodiments. When the filter is actively filtering the ocular artifacts, the correlation values would be low by definition—thus, maximizing this metric alone would mean less filtering of the ocular sources. However, the percentage of ocular artifacts in EEG is considered to be less than the actual EEG signals—thus, increasing the overall correlation value would still serve as a partial metric. A second term is added to the cost function Λ, which is the 2-norm square of the difference between the raw EEG signal and the identified ocular component (this difference is ultimately defined as the clean EEG signal). By minimizing Λ, the amplitude of the cleaned EEG is ultimately taken to be as low as possible, while maintaining high correlation with the raw data due to the first metric. Constant weights 0<[α₁ α₂]<1 for these two partial metrics were also introduced to be able to adjust the level of their contributions. The γ and q values were also bounded, in which the upper bounds are determined to generate a decent filtering performance as examined from an a priori data set. With a running window (w) of 200 ms and 0% overlap, the cost function to be minimized for channel i is defined as:

$${Λ}_{i} = \frac{{α}_{1} * {\sum }_{w = 1}^{n} {‖ {E}_{i}^{w} - {z}_{i}^{w} ‖}_{2}^{2}}{{α}_{2} * {\sum }_{w = 1}^{n} {C}_{i}^{w}}$$

where Cᵢ^{w} is the vector of correlation coefficients for the running windows over all data used for optimization, Eᵢ^{w} is the vector of windowed raw EEG data for channel i and zᵢ^{w} is the vector of windowed identified ocular component that is reflected onto channel i. Finally, ∥Eᵢ^{w}−zᵢ^{w}∥₂² is the 2-norm square of the error term and n is the total number of windows in the data set.

Ocular Artifacts: The system updates an adaptive filter framework in real-time, having the raw EEG signal as the noise contaminated source and the secondary signal as the reference contaminating source (in this case the artifacts). By adapting its weights in real-time, the filter seeks to identify and separate the noise source component that was reflected onto the signal in question (cleaned EEG). Therefore, having a good measurement of the contaminating source (e.g. ocular and motion artifacts) is crucial. It is noted that a well calibrated adaptive framework would effectively eliminate, especially from neighboring electrode locations, neural signal components that may be present in these sources. Applications that require only a few electrode channels (e.g., brain-computer interfaces that rely on EEG signals from mid-central areas), may benefit from the usage of standard channel locations that are close to ocular artifact generators as noise sources, assuming that they share no common neural components. However, for other applications that require a wider coverage of scalp areas, noise profiles are best observed by using dedicated measurement channels. Another disadvantage of using standard electrode locations as noise sources would be rendering these channels obsolete for real-time decoding purposes and discounting the information they would carry for relevant applications.

Eye blinks and eye movements: Eye blink and eye movement artifacts occur due to the electric potential changes around eyes and are one of the main sources of artifact contamination in EEG recordings. They can be dominant in amplitude modulation of EEG channels, and due to volume conduction, can contaminate the entire scalp EEG recordings with varying amplitude and profiles. The noise propagation profile can be highly variable not only across different sessions, but also within a recording session due to experimental conditions such as excessive electrode contact impedance changes due to sweating of scalp, cable tagging/pulling, or simply because of the blink and eye motion potential intensities.

Different methodologies and ocular electrode sites can be used to capture ocular artifacts; however, to the best of our knowledge, there is not a consensus on the sites to be used for this purpose. In some embodiments, a subset of electrodes, either of the EEG or a separate system that is time synchronized, may be placed above and below the eye and/or at left and right temples. In some embodiments, signals that are measured in pairs (for V-EOG and H-EOG) may be utilized to capture a better artifact profile and suppress any common low amplitude components that might be present in the measurement channels, resulting in two noise reference measurements calculated as:

V EOG(t)=V EOG_{U}(t)−V EOG_{L}(t)

H EOG(t)=HEOG_{R}(t)−HEOG_{L}(t)

Low frequency drifts and bias on EEG channels: Real-time brain-machine interface (BMI) applications can benefit from not only the removal of the ocular artifact related components by an ANC filter scheme, but also low frequency drifts and signal biases on EEG channels. Very low frequency drifts can be caused by many factors such as slow electrode scalp impedance changes and amplifier characteristics. Depending on the cause, drifts found in different electrode locations can be uncoupled and uncorrelated. One frequently used method to eliminate this is the low-pass filtering of EEG measurements. Although it is a clear solution for offline applications and signal analysis, under real-time conditions, a low pass filter induces phase (delay) to the filtered signal, dependent also on the filter order and cut-off frequency.

Electrode signal bias is often caused by the measurement system and initial measurement point before data logging starts. Ideally, a neural signal should be logged as a zero mean oscillatory profile. However, due to scalp location dependent artifact, drift and bias characteristics, signals could be recorded with intermittent or continuously drifting profiles with sudden amplitude changes. Again, in an offline setting deriving a zero mean signal does not pose a problem and can be handled with classic filters. However, short drifts caused by ocular noise profiles, or step-like amplitude shifts caused by electrode cable tagging cannot be easily handled and requires an expert investigation in an offline setting. A sample by sample drift and bias removing scheme can also be used for quick removal of these components from EEG source measurements. It should be noted that within this framework, we do not make assumptions on the drift characteristics (i.e. linear or nonlinear, persistent or intermittent). In some embodiments for the ocular cleaning framework, a constant signal with amplitude +1 may be added as an external noise reference to the system, and an additional weight may be adapted to minimize the overall 1 gain per channel, thus eliminating any bias and drift that is present in EEG measurement channels. A full framework with drift and bias removal architecture is provided by the ocular cleaning framework 100 discussed herein.

Adaptive Filter Implementation for Motion Artifacts: Motion artifacts are perhaps the most challenging contaminants of EEG signals to handle. It is generally agreed that motion artifacts vary widely across subjects, and even within sessions. Motion artifacts manifest themselves when there is a relative motion between the electrode tip of an EEG and the scalp. The changes in electrode/conductive electrolyte and electrolyte/scalp interfaces create sudden changes in impedance values, and thus adds associated transient dynamics in the recorded EEG signals. Any additional disturbance due to the continuation of the motion before the previous disturbance reaches its steady-state adds an additional layer of complexity to the problem, as both transients start interacting. We have found these complex dynamics to be highly non-linear, and a statistical or linear framework's effectiveness is not ideal to handle these non-linear projections, as they would at best help to reduce the level of contamination, but significant residuals would remain in the data set. For these reasons, the adaptive framework or methods discussed herein allows for a nonlinear projection. Another difficulty comes from the harmonics of the fundamental contamination frequency (head movement frequency), which are non-existent in neural data. Similar to using the EOG channels as reference signals for filtering ocular artifacts, IMU data, gravity compensated, may be utilized as reference signals for motion artifacts in some embodiments. In some embodiments, the frequency peaks on the vector norm of the acceleration values may be found. Although the acceleration to individual EEG sensor projections are expected to be non-linear, frequency peaks may be found to be very similar to that of the contaminated EEG. In some embodiments, these frequency peaks may be used to generate a narrowband filter-bank. This process is done to generate a new reference signal to target not only the fundamental frequency, but also its harmonics in EEG. The passband boundaries for each filter may be selected as [fⱼ−0.6 fⱼ+0.6] Hz, where fᵢ is the i^{th} frequency peak. Each narrow band filtered reference signal may then be passed through a second order Volterra Series representation.

Volterra series modeling of the non-linear systems is an approach used in many disciplines. For the purpose of adaptive filtering, the model allows us to use the linear adaptive filter formulations. A second-order Volterra series expansion is given below:

$$d ( i ) = {\sum }_{{I}_{1} = 0}^{∞} {w}_{o 1} ( {I}_{1} ) r ( i - {I}_{1} ) + {\sum }_{{I}_{2} = {I}_{1}}^{∞} {w}_{o 2} ( {I}_{1} , {I}_{2} ) r ( i - {I}_{1} ) r ( i - {I}_{2} )$$

Here d(i) represents the unknown representation of the of the motion artifact in the EEG signal, at sample i. r(i) is the reference value used to identify the motion artifact projection, such as IMU data. The terms wₒₖ(l₁, . . . , lₖ), k=1, 2 represents the Volterra kernel to be identified via the adaptation rule. This is a tapped line filter representation, for which taps [1,2,3] may be used. For multiple reference signals, all references were tapped and fed into the Volterra representation. One very important aspect is the selection of the reference signal that is used to identify the motion artifacts in EEG signals. In some embodiments, the acceleration values, after gravity compensation using the quaternion of the IMU, may be used as the IMU data or reference for motion artifacts. In some embodiments, the IMU data or reference may be subjected to a Volterra expansion, such as second-order Volterra series expansion, prior to application of a H^{∞} adaptation rule and combining with incoming EEG data to remove artifacts.

In some embodiments, prior to implementing the motion artifact removal method, the ocular artifacts may be cleaned using our H^{∞} method, implemented on the raw EEG data (FIG. 4). This process results in ocular artifact free, zero-mean data with no drift s₀. In some embodiments, the EEG data may then be zero-phase FIR filtered, e.g. in [0.3-15] Hz range. Observed from their power spectrum, this range of EEG data is found to include all visible motion artifact harmonics. The discussion regarding the higher frequency level contamination is left for further discussion below.

The parameters of the series were identified using the abovementioned H^{∞} criterion. A generic second-order Volterra series expansion is given in (2).

$$d ( i ) = {\sum }_{{l}_{1} = 0}^{∞} {w}_{o 1} ( {l}_{1} ) r ( i - {l}_{1} ) + {\sum }_{{l}_{2} = {l}_{1}}^{∞} {w}_{o 2} ( {l}_{1} , {l}_{2} ) r ( i - {l}_{1} ) r ( i - {l}_{2} ) \qquad ( 2 )$$

Here d(i) represents the unknown representation of the of the motion artifact in the EEG signal, at sample i. r(i) is the reference value used to identify the motion artifact projection. The terms wₒₖ(l₁, . . . , lₖ), k=1, 2 represents the Volterra kernel to be identified via the H^{∞} adaptation rule. This is a tapped line filter representation, for which we have used taps [1, 2, and 3]. For multiple reference signals, all references were tapped and fed into the Volterra representation. Before feeding the EEG signal into the filtering framework, the data was cleaned of ocular artifacts using our ocular cleaning framework discussed above. In some embodiments, all scalp EEG data may then be common average referenced. The EEG signal is then filtered using our method in a cascade manner where the input of the adaptive filtering process j+1 is the output of process j.

BCG/EEG artifacts: Methods that utilize the average template of the artifacts for subtracting it from the measurements are known in neural signal de-noising. The biggest problem for these methods is to eliminate the residual artifacts after cleaning due to the time domain amplitude variations in the signal, and thus (short duration) mismatch of the template and the artifacts. The unified artifact removal framework discussed above may be utilized to accommodate these variations for the BCG signals. Instead of using the common approach, the general H^{∞} method may be utilized, which also provides adaptation to amplitude variations simultaneously. In some embodiments, EEG data may be cleaned of gradient artifact using standard tools. After, ocular artifacts may be cleaned from the EEG data from using our abovementioned method. In some embodiments, an electrocardiography (ECG) average may optionally be calculated using the contaminated channels to serve as a template or reference, if necessary. This template, without further processing, was used as a reference signal to our general H^{∞} framework or method discussed in detail previously above. In other words, some embodiments of the H^{∞} methods may utilize an ECG average provided to a H^{∞} module as a reference to clean a received EEG signal of BCG artifacts, in a similar manner as for ocular or motion artifacts.

tACS/EEG artifacts: The tACS stimulation artifacts may manifest themselves as very high amplitude contaminants (e.g. ˜10⁴ times the expected EEG amplitude). Similar to the BCG removal method or other methods described above, a template of the contamination is generated to be used as a reference signal for the H^{∞} method. In some embodiments, first the nonlinear drifts in a data set may be removed using a 5^{th} order polynomial (e.g. corresponding to lower than 0.1 Hz oscillations). EEG data amplitudes may then be normalized to the [−1 1] range, while saving the scaling coefficients (calculated using a clean segment) for later use. Then each EEG channel data may be smoothed using a Savitzky-Golay filter in some embodiments (e.g. order 7 and frame length of 301). This smoothing is intended to eliminate actual neural signals oscillations superimposed onto the high amplitude artifacts. In some embodiments, a Hilbert transform may then be implemented; the instantaneous phase and amplitude of the signal may be derived; and a synthetic data set that has amplitude of 1 and phase identical to the values derived from the Hilbert transform may be generated. At this point, the synthetic data has no amplitude modulation information, but the phase information matches that of the average artifacts. This phase matched reference may be used in the H^{∞} method to clean the EEG data in a similar manner as discussed previously. Further, the output may be scaled back to original levels using the saved amplitude scaling coefficients.

### Experimental Example

The following examples are included to demonstrate particular aspects of the present disclosure. It should be appreciated by those of ordinary skill in the art that the methods described in the examples that follow merely represent illustrative embodiments of the disclosure. Those of ordinary skill in the art should, in light of the present disclosure, appreciate that many changes can be made in the specific embodiments described and still obtain a like or similar result without departing from the spirit and scope of the present disclosure. Generally, the experimental examples discussed herein were conducted with setups similar to embodiments of the systems and methods discussed above.

Results Ocular Artifact Removal: Subjects: Three healthy adults, including one with paraplegia due to spinal cord injury, participated in this study. The able bodied subject walked on a treadmill at 1 km/h, whereas the paraplegic and second able bodied participant performed a repeated walk-stance-walk task with the assistance of a powered lower-body robotic exoskeleton (REX Bionics Inc., New Zealand).

EEG Measurements: EEG data were recorded in a real-time experimental session. Experimental protocol, decoding methodology and initial performance evaluation is is not discussed here, but was performed in accordance with prior work. Raw EEG and ocular artifact source measurements were used for the analyses in this study. To capture the dynamically changing eye blink and eye motion profiles, four electrooculography (EOG) electrodes from a 64 channel active EEG electrode cap (actiCAP, Brain Products GmbH) were placed on the head of the subject according to the international 10-20 system having FCz as reference and AFz as ground channels. A wireless interface (MOVE system, Brain Products GmbH) was used to record data, sampled at 100 Hz. Ocular electrodes were placed above and below the left eye to measure the vertical ocular artifacts (PO10 for VEOG_{U} and PO9 for VEOG_{L}), and left and right temples to measure the horizontal ocular artifacts (TP9 for HEOG_{L} and TP10 for HEOG_{R}). Locations of these electrodes in an experimental setting were in accordance with FIG. 1. All electrodes used in this study are active electrodes. Two sets of electrodes used to characterize eye blink and eye motion artifacts. Difference of upper and lower vertical EOG electrodes are used to capture vertical EOG components, and difference of right and left temple electrodes is used to capture horizontal EOG components. Ocular framework 100 shows a real-time adaptive filtering framework for simultaneous eye blink, eye motion, EEG drift and bias removal.

Time domain analysis: Analysis was conducted in accordance with the framework discussed above, particular for ocular artifact removal. The simultaneous real-time removal of ocular artifact, drift and bias was first investigated in time domain analysis using correlation coefficients between raw and processed data. EEG data was logged in DC mode with no pre-processing applied. In the sample data set used in these analyses, channel F7 was recorded having around 4 μV of drift, where the mean signal amplitude was recorded as −1281 μV. This is a rather large mean amplitude recording; however, it is also a good example to test the performance of the proposed simultaneous filtering framework. It should be remembered that the proposed filtering scheme is a real-time sample by sample filter; therefore, it is responsive to local biases and drifts, as well as drifts and biases that occur during the entire data recording. Time course of real-time filtering results are summarized in FIGS. 6A-6C for select channels. These include two frontal electrode location, which are of close proximity to ocular artifact generators (FP1 and FP2), one location which is close to both eye blink and eye motion sources (AF8), one central (CZ) and one parietal location (POZ). For plotting purposes, and to be able to compare the high frequency and low amplitude signal components before and after H^{∞} filtering, raw EEG data (after submitted for H^{∞} filtering) and ICA processed data were zero meaned. However, ASR and H^{∞} filters outputs were plotted with no further processing. The correlation values between raw EEG recordings and H^{∞} filtered recordings were calculated for 200 ms windows with 0% overlap. To be able to assess the correlation values between low amplitude raw and filtered EEG data when ocular artifacts and intermediate drifts and biases present, windowed data was removed of trends prior to calculating the correlation coefficients. This was done to capture the correlation values when the windowed data include rising or falling edge of the blink. Naturally, the filtered version of this raw data segment is expected to be zero mean with no artifact related trends. Thus, detrending was done to correlate the neural EEG data that was driven-by ocular artifacts, given the amplitude of artifacts can be 5 times larger than of the filtered counterpart.

The uppermost plot (shaded) of panel-a raster plot of FIG. 6A represents the measured reference noise sources; the horizontal and vertical ocular artifact components. The plots include time course plots of raw ICA and ASR processed and H^{∞} filtered EEG data, as well as correlation values between raw and H^{∞} filtered data. Black circular marks represent the correlation values which are scaled to have a 100% as maximum, and are marked for the leading 200 ms windows. FIG. 6A's plot is mostly for vertical ocular artifact contamination. As can be seen from the figure, contamination amplitude, shape and polarity can vary dramatically depending on the electrode location due to volume conduction. This 6 seconds section of the contaminated EEG was selected for illustrative purposes, as it contains both regular and irregular eye-blink profiles. The blink in 2-3 seconds interval has a more regular shape (close to Gaussian). In general, methods based on template subtraction for artifact contamination might be able to identify such a profile easier. The general shape of the contaminant on scalp locations can be seen as a scaled version of the reference measurement. For electrode locations away from the artifact generators, the driven neural EEG signals' amplitudes become more visible as the amplitude of the contaminant decreases. An effective real-time method should able to isolate and preserve these neural signals. All three processing methods yield good performances in terms of correlation coefficients for this section of the data set (i.e. 2.5th seconds for AF8, CZ and POZ locations). However, the shape and amplitude of the ocular artifact contamination does not conform to a general representation. This stresses the importance of having a clear measurement of the contaminant in real-time and identifying it's reflection on all electrode locations. The section of the plot between 3-6 seconds is an example of the irregular profiles. This includes superimposed fast blinks and low frequency, high amplitude before/after-blink tailing effects. FP1, FP2 and AF8 raw EEG signals reflect the contaminant's amplitude distribution as they are close to the artifact generators. However, the contamination of CZ and POZ channels are in a more distorted form and cannot be generalized by a single template that would be used for FP1 and FP2 electrodes. The ASR and H^{∞} methods remain effective on removing the artifact components on these channels. This shows the advantage of moving windows application of ASR method and sample-by-sample application of the H^{∞} filter. The ICA method cannot identify all sections of the contaminants, and thus results in a less effective performance compared to the other methods. This is partially due to the ICA's application on the entire data set with fixed weights, thus making it less sensitive to local changes in all electrode locations.

The two inset plots (FIG. 6B) are close up plots of channels FP2 and CZ between 1-4 and 3-6 seconds respectively, and show the regular and irregular contamination profiles and the filtering performances. The advantage of sample by sample filter weight adaptation to fixed weight ICA method is seen clearly on the tailing effects before and after the dominant blink profiles. FP2 electrode raw signal peak is seen around 2.5 seconds. The tailing effect results in negative amplitudes for about 0.8 seconds. The ASR and H^{∞} filter, suppresses this tailing effect and pulls the small, higher frequency oscillations around zero amplitude, whereas this suppression is less in ICA immediately before and after the main blink profile. The CZ inset plot shows the effectiveness of the three methods when the components are reflected in a distorted form on mid and parietal locations. For this section of the data, the ASR and H^{∞} methods clearly surpass the ICA method. The maximum raw signal amplitude was registered as 60.37 μV due to artifact contamination. ICA pruned signal results in an amplitude of 48.22 μV, whereas the ASR processed and H^{∞} filtered signal amplitudes are 13.39 and 14.93 μV respectively.

The V-EOG profile shows another very low frequency and low amplitude artifact, starting as a linear trend around the 1st second and peaking at 1.7 seconds. This lower amplitude contamination has visible components only on very frontal electrodes (FP1 and FP2). Both the ICA and H^{∞} methods are successful in removal of this component, however the ASR method does not clear this EEG section, which is contaminated by this low amplitude artifact. Also for channel AF8, the ICA and H^{∞} methods keeps the EEG data intact in between 0-0.8 seconds, which has a minimum of −23 μV (which can be seen as a low-frequency neural signal, and is also not present in EOG source measurements). The ASR method however, filters this section and suppresses the low frequency components. This brings the importance of being selective of what is filtered in a raw EEG recording. The ICA method relates ocular artifact components to frontal electrodes, thus is selective of the ocular artifacts. However, it suffers from the identification of shape-distorted components on electrode locations away from artifact generators. The ASR method covers this problem, however it is not as selective compared to the ICA method on ocular components. The H^{∞} filter takes advantage of the reference signal measurements and is very selective in that sense. It also covers the local changes on all electrode locations by its sample adaptive formulation, and inherently robust by definition of minimizing the error to disturbance 1 gain.

Finally, FIG. 6C shows successive vertical and horizontal EOGs. In this section of the data set, the horizontal EOG component is superimposed to the tailing effects of blinks. The contamination is most visible in raw EEG signals of FP1 and FZ electrodes, resulting in sharp amplitude changes of around 45 μV. Again the ASR and H^{∞} methods perform in similar levels, filtering both the superimposed tailing and horizontal EOG components from raw EEG recordings.

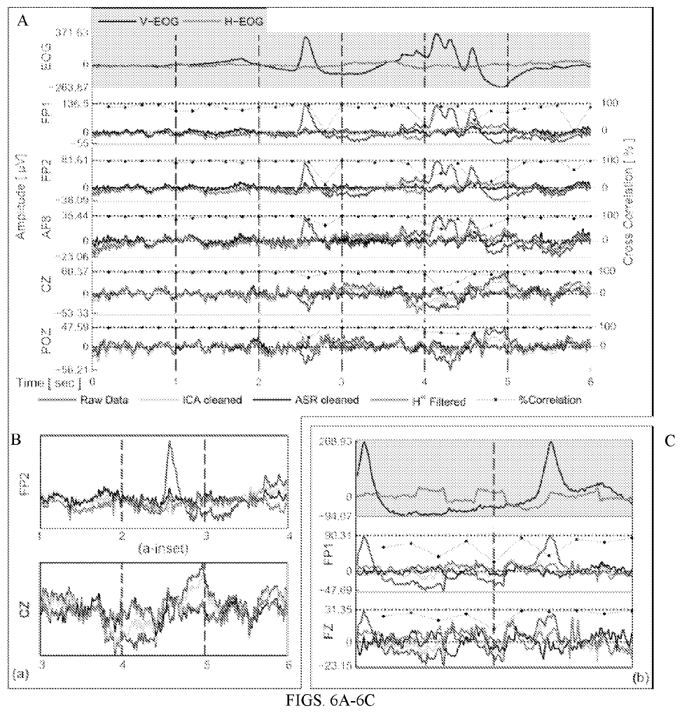

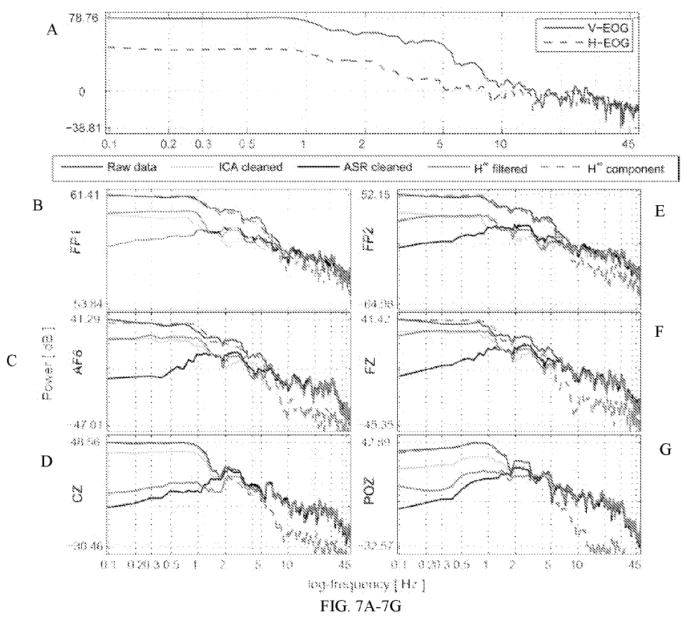

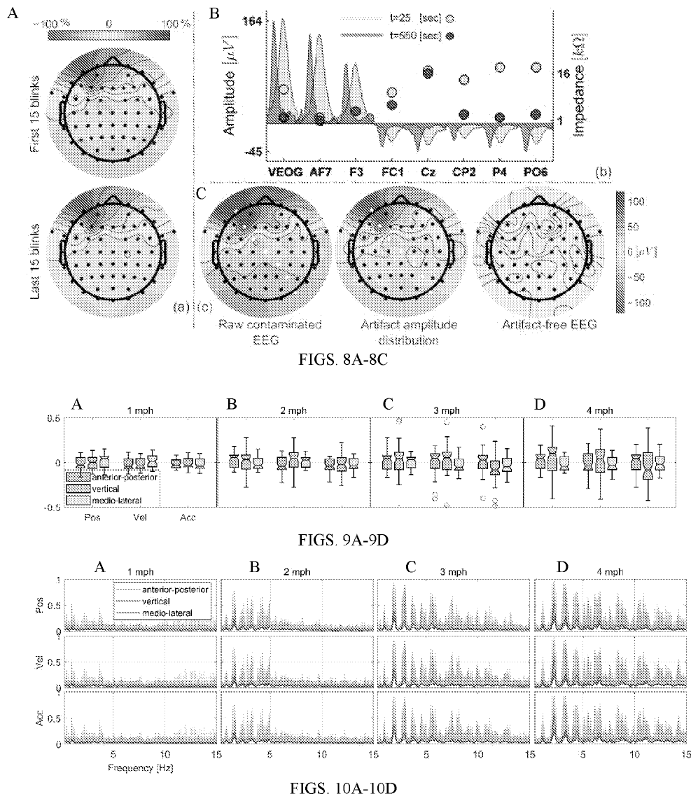

Frequency domain analysis: FIG. 7A shows the measured V-EOG and H-EOG reference power spectra, having high power in low frequency bands (less than 8-10 Hz) and below 0 dB for higher frequencies. FIGS. 7A-7G are complementary to the time domain analyses and the same sections of the data were used for better comparison. FIGS. 7A-7G show power Spectral densities of VEOG, HEOG, raw EEG, ICA, ASR and H^{∞} filtered data as well as identified ocular components by the H^{∞} filter. Power decrease in low frequency regions is seen due to filtering of ocular artifacts. High frequency components are left intact for both real-time H^{∞} filtered and offline ICA and ASR processed data. The power spectral plots of select channels (FIGS. 7B-7G) show similar suppression in frequencies less than 8 Hz for FP1 and FP2, and less than 5 Hz for the remaining electrode locations for all three methods. Higher frequency components for all 3 data sets remain in close correlation with the raw signal power spectra. In general, there is a higher power drop of the H^{∞} filtered data for less than 2 Hz frequency band compared to the ICA method, due to the removal of higher amplitude components of these electrode measurements. The suppression is even larger in ASR processed data, showing similar power spectra for all channels, having close to zero power in around 0.1 Hz and an increasing tendency until around 4 Hz. This could be due to fixed low-pass and high-pass parameters one needs to set before the application of ASR method and could yield different profiles for different values. The low frequency suppression of the ASR method (summarized in Time Domain Analysis section) for channel AF8 (0-0.8 seconds) is also visible in frequency domain analysis, where the ASR processed data registered having negative dB power in less than 0.5 Hz region, and ICA and H^{∞} methods having similar power spectra for low frequencies. The H^{∞} filter power spectral characteristics shows more variability depending on the electrode location, especially for very low frequencies. As an example, the H^{∞} filtered data has a similar power spectral density profile compared to the ICA method for channels FP1 and AF8, however it is closer to the ASR method for channels CZ and POZ. One reason for this change is the sample adaptive weight profiles of the H^{∞} filter. The other reason is the optimized γ and q parameters for different electrode locations instead of using fixed parameters for all channels (summarized in Materials & Method section). The power spectra of the blink components reflected onto channels identified by the H^{∞} filter are also shown in these plots. One can notice the similarities below 5 Hz compared to the raw EEG data. This is expected as the ocular artifacts are found dominant in amplitude for all channels and the frequency characteristics conform with the measured ocular reference sources (also summarized in FIG. 8A-8C). Sudden power drop after 5 Hz frequency of the identified components conforms with the fact that the ocular source measurements include none to minimal neural activity as a result of using dedicated electrodes for source measurements.

Processed signal properties: The ICA method relies on the weight identification to form a mixing matrix to determine unique components in the data set. The H^{∞} filtering method used is also formulated around the weight identification problem, having the assumption of time variation of the weights and also robustness properties. The sample-adaptive application results in continuously adapting weights for all channels. The ICA identified fixed weights and the time varying H^{∞} weights are compared in FIG. 5. Values are complimentary to the time traces reported in FIGS. 6A-6C. After converging to their optimal values (which takes around 200 ms), the H^{∞} weights are adapted to the changing properties of the ocular artifacts and drifts and biases. In FIG. 5 (inset), electrode locations are grouped to four Region of Interests (ROI) as Pre-Frontal (PF), Frontal (F), Central (C) and Parietal (P) regions. Weights of the electrodes in these regions are plotted first left-to-right, then front-to-back as shown on the inset electrode location map. The red-solid line shows the ICA identified weights, and blue-solid line shows the mean H^{∞} filter weights. The blue shaded region represents the interval of weight change around its mean. All values are normalized to have a better comparison. The weight change interval of H^{∞} filter is very narrow for the frontal electrodes, where the contamination is most dominant. H^{∞} weights and ICA weights are very similar for PF electrodes as the contamination is dominant in this region. Time variation of H^{∞} weights gets larger for F, C, and P regions as the filter adapts to varying levels of contamination amplitude and profiles, thus resulting in a better filtering performance. The method actively seeks correlation with the noise source measurement and the EEG channel amplitude, thus can adapt to the less visible or shape distorted contaminations better. As noted in FIG. 5, the weight change intervals are much larger in other locations compared to the frontal areas, which results in a dynamic capture of ocular artifact contamination.

Inset histograms of FIG. 5 compare the raw EEG (red) and H^{∞} filtered EEG (blue). Raw EEG signals show high kurtosis and some levels of skewness when contaminated by artifacts. The filtered EEG data show more distribution around zero mean compared to raw EEG data. Table 1 summarizes the skewness and kurtosis values for all three methods for the entire 10 minutes of data recorded. Also the mean correlation coefficients of data across all EEG electrodes and 335 moving windows processed by all three methods and the raw signal for the entire (10 minutes) of recorded EEG is summarized in table 2. High correlation value of the ICA processed signal vs. raw EEG signal, when all electrodes are considered, can be due to less sensitivity to ocular artifact contamination for channels away from ocular sources (summarized in FIGS. 6A-6C).

TABLE 1
Comparison of kurtosis and skewness of the raw EEG, ICA processed EEG, ASR
processed EEG and H^{∞} filtered EEG data sets

| | Raw EEG | | ICA Processed | | ASR Processed | | H^{∞} Filtered | |
|---|---|---|---|---|---|---|---|---|
| | Kurtosis | Skewness | Kurtosis | Skewness | Kurtosis | Skewness | Kurtosis | Skewness |
| FP1 | 9.7496 | 2.0559 | 3.8615 | 0.1627 | 4.1965 | 0.1184 | 3.4721 | 0.2589 |
| FP2 | 8.1662 | 1.7418 | 3.6995 | 0.2766 | 4.0007 | 0.1199 | 3.7236 | 0.2620 |
| AFS | 4.4459 | 0.5942 | 3.5550 | −0.0498 | 3.3859 | 0.0706 | 3.5552 | 0.0202 |
| CZ | 3.6154 | −0.0125 | 3.2881 | 0.1319 | 3.3712 | 0.0128 | 3.4694 | 0.0460 |
| POZ | 4.0164 | −0.1691 | 3.5666 | 0.0970 | 4.0337 | −0.0190 | 4.2440 | −0.0033 |

TABLE 2
Mean correlation coefficients across all EEG channels and 5 sec
moving windows (for 10 mins of recording) with 90% overlap

| | ICA | ASR | H^{∞} |
|---|---|---|---|
| RAW | 0.8549 | 0.6711 | 0.6871 |
| ICA | | 0.7413 | 0.7774 |
| ASR | | | 0.7854 |

Volume conduction and topographical maps of the contaminants: Effective filtering of the artifacts from EEG signals refers to identification of artifact components that is reflected onto the EEG signal to be recovered. Referring to equation 5, the artifact components can be derived as rᵢ^{T}ŵᵢ for reference measurement r and estimated weight ŵ at sample i. This gives the opportunity to dynamically examine the artifact component distribution over scalp areas at any given time point. Summarizing these analyses, FIGS. 8A-8C provide 3 panels of scalp topographical plots of EEG contamination, time traces for select electrodes and components of eye blinks on select electrode locations (identified using the H^{∞} filter). FIG. 8A was plotted to examine the average ocular artifact contamination or percentage of contamination over scalp areas to the characterized VEOG component, where the upper plot shows scalp distribution of the mean values of 15 eye blinks near the beginning of the session, and lower plot shows the mean of last 15 eye blinks for a 10 minutes data collection session. As mentioned before, our data collection session includes repeated walk and stop cycles for about 10 minutes. 15 eye blinks from the second set of walk task were manually selected, and another 15 eye blinks from the last set of the same task. The reason for selecting two sets of blink profiles from the beginning and end of the data is to visualize the effect of impedance changes on the artifact contamination profiles. These artifacts were averaged to rule out the possibility of comparing lower intensity vs higher intensity blink profiles. Nevertheless, two single blink profile comparisons were made in FIG. 8B. This panel also shows the amplitude and impedance changes at the start (blue color) and end (cyan color) of the experimental session. Plots were prepared for a select number of channels that cover the scalp areas diagonally (marked white on FIG. 8C). Blue and cyan circles are impedance values recorded at the beginning and end of the experiment, respectively. Finally, FIG. 8C compares a single blink peak amplitude over all scalp regions. Left topographical plot shows the raw EEG data amplitudes and middle plot is only the identified contaminants' amplitudes. High similarity in between two plots suggests a high level of contamination over all scalp areas in both amplitude and scalp distribution. Right topographical scalp map shows the EEG amplitude distribution after cleaned of ocular artifacts using the real-time H^{∞} filter.

The impedance values at the beginning of the session, having a maximum of 18 kΩ for select electrodes, were higher compared to the impedance values at the end of the session. In FIG. 8A, the area under curve of each measured ocular artifact profile was calculated and its percentage reflection as identified ocular artifact profiles was reported for each channel location (where negative values represent polarity change). Upper topographic plot was derived for the beginning of the session and the lower one for the end of the session. The average percentage area under curve distribution is very similar in these two cases. Experimental protocols provided that all impedance values be under 30 kΩ. A major amplitude percentage change in contaminants was not seen that is in direct correlation with the impedance changes. Channel AF8 shows average component change of 6.57% with an impedance increase of 8 kΩ and channel F6 shows a 2.9% increase when the impedance remained constant. The maximum impedance decrease was seen in channel CP4 (24 to 1 kΩ) and the amplitude remained relatively unchanged (1.29% decrease). These are relatively low impedance changes, and according to, are not expected to have major amplitude changes especially under cool and dry experimental conditions. Nonetheless, FIG. 8B summarizes the identified component distribution of a set of scalp-diagonal electrodes for single blinks. The first profile is the measured VEOG component, followed by its reflection on diagonal scalp areas. As the impedance drops through the end of the session (blue marks), the amplitude of the identified components increased for these select electrodes and also for the VEOG recordings. One property to notice from both FIG. 8A-8B plots is the variability of amplitude distribution of VEOG components over scalp areas. A clear pattern was not observed, such as a decreasing amplitude as electrode locations are further away from ocular sources, even for cases where the impedance values are comparable. This further justifies the importance of an artifact removal method that can actively seek EOG reflections on all electrode locations.

FIG. 8C shows the signal amplitude distribution (in μV) when a blink's amplitude is at its peak. These topographical maps show the level of the measured raw EEG amplitude information, which are as a result of ocular artifact contamination. Left shows the topographical plot of the raw EEG amplitudes. Middle plot shows only the identified blink component's amplitude. There is a high similarity between the two in terms of amplitude information and scalp distribution. Thus, it is safe to conclude that an EOG artifact can greatly affect almost all the scalp areas, and should be taken into account especially for real-time closed loop BMI applications. The right topographical plot of FIG. 8C is the EEG signal amplitudes after filtered by an H^{∞} filter. As expected, the majority of the scalp signals are around ±15 μV and are more likely to be task related neural sources.

Discussion: The real-time applicability and performance of an artifact removal technique is of great importance to BMI applications. EEG signals are inherently prone to artifacts with different amplitude and frequency characteristics. A robust artifact removal technique is proposed that removes ocular artifacts, DC shifts and biases. These artifacts are present in almost all experimental sessions, and as shown, dominant in EEG measurements in both time and frequency domain. An H^{∞} ANC framework was developed to remove eye blink, eye motion and EEG signal bias and drift simultaneously using a real-time sample adaptive formulation. Successful removal of artifacts suggests a good identification of the contaminant components that are superimposed onto the neural EEG signals. Analysis of these components over all scalp areas gives a clear picture of the contamination spatial distribution due to volume conduction. It has been found that, when the blink amplitudes become dominant, the raw EEG and the contaminants alone share similarities both in amplitude and spatial distribution.

The presence of such dominant artifactual components can hinder the true performance of a real-time BMI decoder. Analysis on the power spectral densities of the identified contaminants also shows similarities in low frequency regions. These results suggests that during the ocular contamination, amplitude, frequency and spatial scalp distribution based features, that are extracted from EEG signals for decoding purposes, share the same adverse effect from ocular contaminations. In the present study, the raw EEG recordings were filtered of ocular artifacts and drifts/biases. In general, decoding methodologies that focus on frequency bands less than 8 Hz could be highly affected by ocular artifacts as even with a bandpass filter used to capture slow cortical oscillations, the ocular artifacts would have high magnitude components in the lower band EEG activity. The present study addresses the negative effects of ocular artifacts for any real-time implementation of BMI decoders. A BMI designer that focuses on these frequency bands has limited options under these circumstances. First, one could design a decoder offline after pruning the EEG signal clean of artifacts. In this case the real-time performance of the same decoder would be adversely affected by these unmodeled artifacts. Second, one could dismiss the frontal electrode locations that are close to ocular sources for real-time decoding, however our analyses show that the contamination amplitude and scalp distribution is dominant in almost all electrode locations. Another option would be not to use the decoder output as a BMI control signal when the blinks are present. However, further analysis on the data set shows over 12.4% ocular artifact contamination in a 10 minute data collection session. Ocular artifact profiles are also subject specific, and occurrences are not periodic or predictable. One interesting result of the analysis is that the ocular artifact densities can also be task specific. As summarized in the prior discussion, the data collection session includes periodic walks and stops with a lower body exoskeleton. It was found that among the 12.4% contamination, the blinks of dominant amplitudes registered as 64.3% when the subject walks, and 35.7% when the subject is in stance position. And more interestingly, the densities of the blinks among the walk sections of the data show an increasing trend as session progresses. This of course cannot be generalized to a wider subject population at this stage; however the results show that the possibility of having such a pattern greatly hinders the applicability of real-time decoders in the presence of ocular artifacts. The highly dynamic nature of these artifacts also suggests that the usage of the same decoder for multiple sessions can also cause the wrong conclusions about the decoder performance.

The comparisons with offline applied ICA show the importance of optimization of filter performance parameters per electrode location and also the local search properties of the artifact removal tool. ICA identified ocular components usually relate to the frontal electrode locations and may be removed by an expert user or an automated method accordingly from all electrode locations. This analysis show that the artifact projection onto electrode sources can vary greatly due to volume conduction, and the definitive properties of the artifacts (such as its profile or amplitude modulation) can be reflected as a distorted version on electrode locations away from artifact generators (such as CZ and POZ electrodes, see FIGS. 6A-6C). The performance of the ICA method reduces in such occurrences, resulting in cleaned EEG amplitude close to the artifact contaminated raw EEG amplitudes.

The overall performance characteristics of the offline applied ASR method are close to the real-time implemented H^{∞} method discussed. Similar to the sample search structure, it uses a moving window of EEG data, therefore actively searches for localized artifact contamination. The problem, however, is the selectivity of the removed/suppressed components from raw EEG recordings. The ASR method uses threshold values to identify any undesired occurrences in EEG. The analysis showed that the EEG components having −23 μV amplitude, which is also not present in the raw EOG measurement are also removed from EEG data (FIG. 6A channel AF8). However, a lesser amplitude (11 μV) section of the EEG, which is a reflection of recorded raw EOG artifact, was not removed. Having explicit definitions of the quantity to be removed, the framework actively seeks the projections of the artifactual signals and drift/bias components on all scalp locations with optimized parameters, and inherently robust to local unexpected changes that might affect the EEG recordings.

Application of this framework to other types of artifact contamination in EEG recordings, such as movement related artifacts, is possible and should be carefully formulated as discussed herein.

Our results show over 95-99.9% correlation between the raw and processed signals at non-ocular artifact regions, and depending on the contamination profile, 40-70% correlation when ocular artifacts are dominant. We also compare our results with the offline Independent Component Analysis (ICA) and Artifact Subspace Reconstruction (ASR) methods, and show that some local quantities are handled better by our sample-adaptive real-time framework. The proposed method therefore allows real-time artifact removal for EEG-based closed-loop BMI applications and EEG studies in general, thereby increasing the range of tasks that can be studied in action and context while reducing the need for discarding data due to artifacts.

Results of Motion Artifact Removal: Subjects: 11 healthy able-bodied adults with no known gait deficiencies participated in this study after giving informed consent. FIGS. 1-2 respectively show the system setup and workspace of the movement data collection setup. The treadmill was surrounded by a motion tracking system, for which the approximate capture area was limited to the head of each subject. Cameras were calibrated before each session.

Subjects were asked to walk on a treadmill (FIG. 2) at 1-to-4 mph speeds for 6 minutes continuously, as 4 different tasks. Each task was started and ended with a 1-minute quiet standing. The walking area was surrounded with 12 OptiTrack motion capture cameras. The workspace of all the cameras was limited to the head of the subjects for increased resolution (mean tracking error ˜0.07 mm).

All subjects were equipped with a 64 channel gel based EEG system (actiCAP, Brain Products GmbH) with active electrodes and 10-20 distribution. 4 electrodes were placed around the eyes of the subject to measure the ocular artifacts in bipolar configuration (TP10-TP9 for Vertical-EOG and P010-P09 for Horizontal-EOG). Peripheral electrodes FT9 and FT10 were moved to FPz and FCz locations accordingly for a denser scalp coverage. Reference and Ground electrodes were moved to the ears. FIG. 1 shows an example of the setup. All subjects were also equipped with 9-axis wireless IMU sensors (APDM, Opal), one at the forehead of the subjects, one on the right foot and one on the left foot. The forehead sensor was used to assess the usability of the IMU information to characterize and/or remove the motion artifacts. The sensors on the feet were used to segment the gait phases. The inset figure (lower-left corner) shows all scalp locations where the infrared markers were placed.

No external layer on EEG electrodes was used (i.e., a medical mesh). An external mesh, covering the electrodes is a standard setup item, as it was found to dramatically reduce the motion of the electrodes and cable sway. For the scope of this work, however, a clear presence of motion artifacts was needed for us to be able to characterize and assess the validity of our cleaning method (FIG. 1 Figure).

5 of the select electrodes (Fz, Cz, Pz, C5, C6) were equipped with different reflective marker configurations, each containing 4 markers. Markers were placed on a transparent plastic medium with negligible weights and attached to each sensor. The surface of the forehead IMU sensor was also equipped with a reflective marker configuration (FIG. 1).

The EEG (1000 Hz), IMU (256 Hz), and OptiTrack (120 Hz) data were synchronized with a manual logic signal at the run-time. For the velocity and acceleration estimation from the optitrack system, per electrode, we have used a linear Kalman filter with a second order nominal model and optimized the model parameters using a constrained optimization tool having the IMU acceleration as an input (the fmincon function in Matlab®).

Non-linear properties of motion artifacts: Analysis was conducted in accordance with the framework discussed above, particular for motion artifact removal. The application of linear correlation analysis alone to gauge the transmission of motion artifacts to EEG signals can hinder the true level of the artifact contamination. In a gel-based EEG setup, the disturbance caused by the movement between the skin-electrolyte and electrolyte-gel interfaces can be a repetitive action, manifesting the artifacts having repetitive transient dynamics. Coupled with the electrode cable bundle and associated sway dynamics, we expect the projection of the artifact from the actual head kinematics to the EEG recordings to be inherently highly non-linear. We would also expect extensive differences on the projection levels of the motion artifacts for different electrode sites. The non-linearity and dynamic characteristics could be reduced to more manageable levels by the use of a head-mesh and limiting the cable sway, essentially coupling the electrodes mechanically. However for the purpose of this study, we have avoided any application that could reduce the artifact contamination and an utilized an EEG setup that is a standard implementation along the lines of the most generic uses.

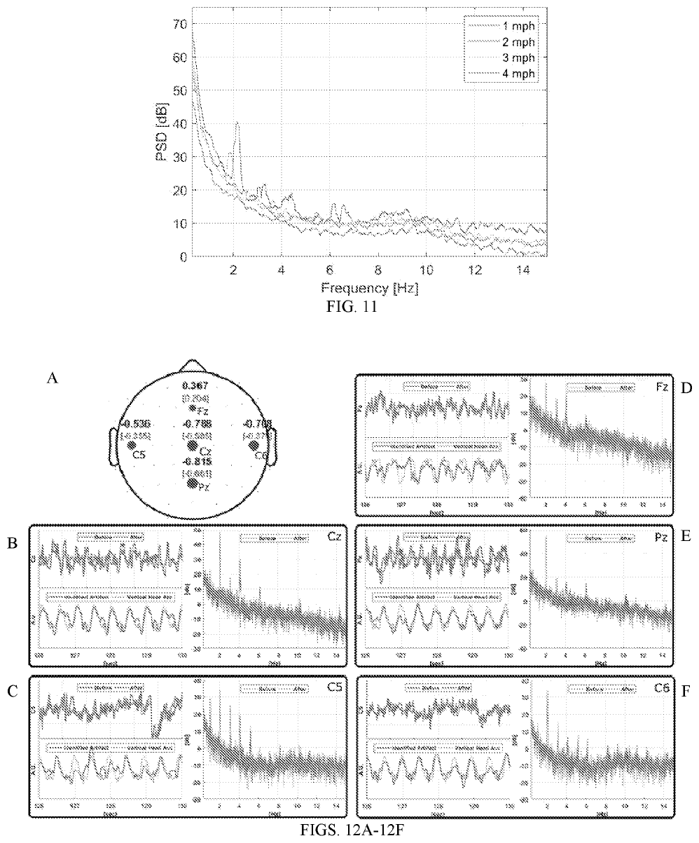

The linear correlation between measured quantities and the EEG signals are summarized in FIGS. 9A-9D. FIGS. 9A-9D show group correlations for all subjects and marked electrodes for different walking speeds. Linear correlation is calculated between the EEG data and the individual sensors position, velocity and acceleration as measured and calculated by the motion capture system. We have grouped all subjects and all marked electrodes and used the entire duration of the experiment to calculate the values. Signals are also shifted where applicable to yield the larger correlation values. The correlation between the EEG data and the measured position of that individual electrode increases as the walking speed increases. In general, the faster speeds yield larger correlations, as well as larger span in vertical movements. However, there are instances of larger median values in anterior-posterior and medio-lateral directions for various speeds and quantities. This shows the dynamic characteristics of the contamination and justifies the use of multi-axis reference signals for cleaning the EEG data. The velocity and acceleration derived from the measured position via a parameter optimized Kalman filter yield similar correlation values, but slightly less compared to the position of the same electrode. The higher speed acceleration data in the vertical direction is found to be inversely correlated with the EEG data. The maximum median value for the given quantities is around 0.1, whereas the overall maximum is ˜0.4. At first glance, these correlation values suggest minimal motion artifact contamination. However, the frequency domain analysis paints a different picture. FIG. 11 shows the grand average PSD of EEG data from all marked electrodes, for all subjects and 2 minutes of continuous walking. Although the spectral motion contamination is inconclusive at slow speed of 1 mph, at higher speeds, the contamination is apparent, exhibiting also strong harmonics. Checking the frequency coherence of the EEG to the EEG electrode kinematics (position, velocity, and acceleration of the individual EEG sensor) supports the idea of strong contamination in higher speeds. FIGS. 10A-10D summarizes the coherence values for all marked electrodes and all subjects. FIGS. 10A-10D shows group coherence for all subjects and marked electrodes for different walking speeds. The median values per axis are plotted as bold lines, and the variation around it, for all subjects and electrodes were plotted as shaded regions. The solid lines represent the mean coherence per axis of motion, and the shaded regions represent the variance around it. As expected, higher walking speeds result in higher coherence values. The differences between position, velocity, and acceleration based synchrony is harder to see in a coherence plot and all quantities exhibit similar peaks and harmonics. One interesting finding is on the existence of coherence peaks at different harmonics. In general, all 3 axes (anterior-posterior, vertical and medio-lateral) of electrode movements yield strong harmonics, the vertical axis usually being the strongest. However, some harmonics appear to be majorly generated by only one or two of the axes. Furthermore, the existence of high amplitude frequency locked components in EEG data (spanning almost 25 dB range in high walking speeds, FIG. 11), and loosely correlated time domain data to position, velocity, and acceleration of the specific EEG sensor suggests a complex non-linear relation between the electrode kinematics and the projected contamination.

Therefore, for a conclusive test of whether or not the EEG is under the influence of motion artifacts, and if there is contamination, the level and time domain characteristics and even the discrete vs continuous existence should be investigated by methods that allow for sample by sample non-linear mapping. This essentially suggests identification of the artifactual components. We investigate the applicability of identifying the nonlinear representation (Volterra Kernel coefficients) through a sample adaptive filter to characterize the motion artifacts. This sample-by-sample adaptation, contrary to the statistical methods, cannot only tell us of the existence the motion artifacts at different instances in a session, but it can also help us define the abovementioned characteristics.

Removal of motion artifacts: As detailed in the previous section, the nonlinear characteristics of the artifactual signal require methods to assess the level of contamination as well as the characteristics of the EEG motion artifacts. The V-H^{∞}/TV formulation allows for the non-linear projection from the reference sources to the contaminated signals (EEG). To test the effectiveness of the method, we have used an individual electrode's (Pz) position, captured by the OptiTrack motion capture system for 4 mph subject walking speed. The fastest walking speed is chosen to ensure the presence of motion artifacts.

The reference signal to the adaptive filter plays fundamental role in removing the artifactual components. For the purpose of this work, we have tracked the precise positions of 5 electrodes for different subjects and motion speeds. For our offline analysis, the availability of this detailed information is valuable in understanding the motion artifact components of the measured signals. However, for a wide-scale application, this information is unlikely to be available, thus we seek to understand the characteristics of the artifacts and determine if another measurement modality can be used instead of the precise electrode position. To accomplish this, we have first used the 3-axis position of the individual sensorized electrodes to remove their respective artifactual components. The position measurements provide us with the oscillatory (regardless of being continuous or intermittent) electrode dynamics, which is technically the main cause of the artifacts (electrode position shift causing the disturbance in the skin/electrolyte and electrolyte/metal interfaces). As summarized in FIG. 4, we have first used the ocular artifact, bias and drift cleaning method to denoise the raw EEG. The signal is then bandpass filtered in 0.3-15 Hz range, and referenced to the common average of all electrodes. To identify the base and harmonic frequencies of the contaminating movement with peak spectral power, the movement signal is spectrally whitened and the peaks are identified. This whitening is done to automate the detection of the spectral peaks with improved accuracy. The reference signal is then bandpass filtered to each of the identified frequencies fⱼ (j=1, . . . n), where n is the number of frequency peaks. The passband boundaries are selected as [fⱼ−1 fⱼ+1] Hz. The EEG signal is then filtered using the V-H^{∞}/TV formulation in a cascade manner where the input of the adaptive filtering process j+1 is the output of process j.

The final clean EEG signal (sₙᵢ) is then used to calculate the non-linear projection of the movement reference to the specific EEG channel, for all samples i as: aᵢ=s₀ᵢ−sₙᵢ, where sₒᵢ is the ocular, bias and drift cleaned EEG. The properties of aᵢ is very informative on the identified artifactual signal characteristics.

FIGS. 12A-12F shows the linear correlation values for the motion tracking equipped sensors w.r.t the vertical axis of the head acceleration measured by the head IMU sensor for 4 mph walking speed (on average, the vertical axis correlates most for this walking speed as shown in FIGS. 9A-9D). FIGS. 12A-12F show identified artifacts and their properties. Scalp values show the linear correlation values between the identified artifact and the vertical forehead acceleration for sensors equipped with the motion tracking markers (full session data—6 minutes). The values in brackets show the values for the raw EEG and same vertical acceleration for comparison. Each panel of plots for different electrodes show the before/after filtering of the motion artifacts for continuous 4 seconds of data for visibility (upper left), the identified artifact of the specific channel and the vertical forehead acceleration (bottom-left), and the power spectrum of the raw EEG and the motion artifact filtered EEG for the whole duration of the experiment (6 minutes) (right). The reported linear correlation values were calculated by shifting the signals to yield the maximum correlation. Negative values in the plots refer to delaying the identified artifacts. The shift data points lie within 50-to-90 [ms]. Top right scalp plot reports the correlation values. Each reminder panel of plots show a short segment of signal (for visibility) of raw and clean EEG (top-left), identified artifact and the vertical head acceleration (bottom-left), and the power spectrums of raw and cleaned EEG signals (right, for the full data set, 6 minutes of recording). Note that the identified artifact vs head acceleration plot changes the sign of the acceleration data for convenience (C5 electrode in the plot). First to notice is the motion artifact contaminated EEG signal and the power of the major artifact peak, locked to the walking speed, and its harmonics. The overlayed clean EEG power spectrum show a clear suppression on the major contamination frequency and all of its harmonics for the given frequency range. This not only shows the effectiveness of the method, but also clearly indicates the selective nature of the cleaning paradigm. The spectral details where the artifact peaks are not visible are an exact match between the raw and cleaned EEG signals. Also, in time domain, EEG amplitude modulation based features are preserved, while only the artifactual components of the signal are cleaned. The keyword—time domain feature—used here does not necessarily limit to the task dependent cortical oscillations and associated amplitude modulations. It rather points to very fine details of an EEG signal's characteristics, and their preservation by our method during motion artifact cleaning of a heavily contaminated EEG signal.

Furthermore, the time domain modulation of the identified artifact signals closely resemble vertical head acceleration values measured simultaneously with the EEG. To summarize the significance: the precise measurement of the position of each electrode were mapped via a nonlinear transformation to the measured EEG signals. This final position projections were then linearly correlated to the IMU measured head acceleration signals. It's found that the identified nonlinear transformation yield high linear correlation values compared with the acceleration sensor data. The linear correlation between the raw EEG and the head acceleration, without any processing, is found to be small, compared to what has been transformed from the position data. An acceleration sensor is a very low-cost device which is already an integral part of many commercial EEG systems. Thus, accessing the synchronized acceleration data from any of those systems poses no difficulties. Even systems without the acceleration sensors can be used by placing an external sensor to the forehead of the subject and synchronizing the data with the EEG signals, as has been done in this study. The high linear correlation between these quantities is a promising prospect to use the acceleration values for a sample-by-sample, real-time cleaning of EEG signal from motion artifacts. One very important property of using the forehead acceleration (IMU) sensor measurements as a reference signal is that it allows for filtering the entire scalp EEG locations. We would also like to stress that the values reported are linear correlations. Therefore, with high levels of confidence, we can say that the usage of acceleration sensor values as a reference input to the adaptive noise canceller is possible. The projection of the forehead acceleration values, via our nonlinear method, to identify a projection that is already highly linearly correlated with the acceleration values should yield similar or better results.

Another observation, confirming the literature on movement artifact removal methods as discussed, is the variability of the identified artifacts by channel locations. The contamination levels (as judged by the spectral peaks and harmonics) are also variable by sensor location as expected. The time domain characteristics of a single channel artifact within the same recording session also show variability. Sample adaptive formulations in this sense allow us to identify informed projections with varying levels of contamination in time domain.

Our next step is to use all 3-axis head acceleration values (measured by the forehead IMU and gravity compensated) as reference signals to our framework and compare the cleaning performance and linear correlation values with the cleaning using position reference.

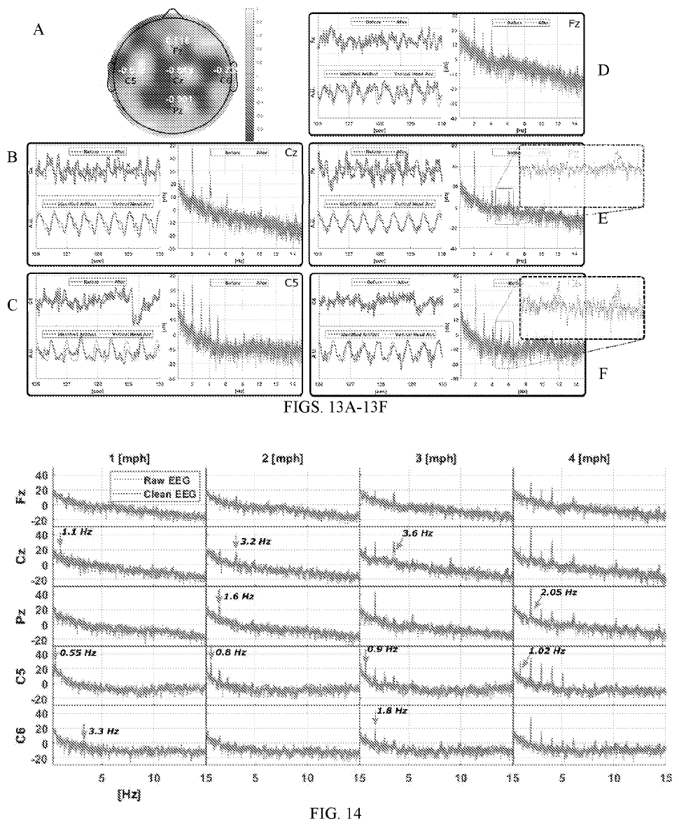

Note that by using the 3-axis acceleration values as a common reference to all EEG locations, we are able to plot a scalp distribution of values. FIGS. 13A-13F show scalp topographical plot showing the distribution of how well the identified artifact signal, per electrode, is related to the head acceleration values via linear correlation. FIGS. 13A-13F identified artifacts when the reference signal is the 3-axis gravity compensated acceleration values. The scalp values show the linear correlation between the identified artifactual signal and the vertical head acceleration. Note the possibility of visualizing values for the entire scalp contamination. Each panel of plots, for select electrodes, show the raw and clean EEG signal segment (upper-left), identified artifact and the vertical head acceleration segment (lower-left) and the power spectra of the raw and clean EEG for the entire data-set (right). Compared to the position reference signal for cleaning, the linear correlation yields higher values for the marked electrodes. This supports the idea of using acceleration as a reference signal, not necessarily because it's by default correlated to the raw EEG, but because its time domain properties and information-rich structure are highly relevant to the motion artifact contamination, should a proper non-linear mapping technique is used for identification.

Another important property to notice is the distribution of relevance among scalp locations. Apparently, as stated in the introduction, due to the high dynamic interaction between the cable bundle and the electrode, bundle sway and other factors that cause non-linear mapping, the contamination level, and correlation sign varies greatly for all scalp locations, and does not follow a clear distribution. The power spectral comparison of raw and clean EEG signals show a very effective cleaning process, which is selective of individual signal contamination level. We would like to stress that the same reference signal and frequency bins were used for all scalp areas as a reference signal. Comparing the harmonic peaks appearances and the prominences, it is clear that each electrode experiences different non-linear contamination dynamics. As an example, the Cz raw signal peaks show a sparse distribution compared to say the C5 and C6 electrodes and the ˜5 Hz harmonic peak is not visible in Cz, Fz or in Pz. Yet the robust adaptive nature of our framework is capable of identifying when there is a relevant peak and when there is not, keeping the frequency information in the signal intact when needed (see the inset plots for C6 and Pz in FIGS. 13A-13F).

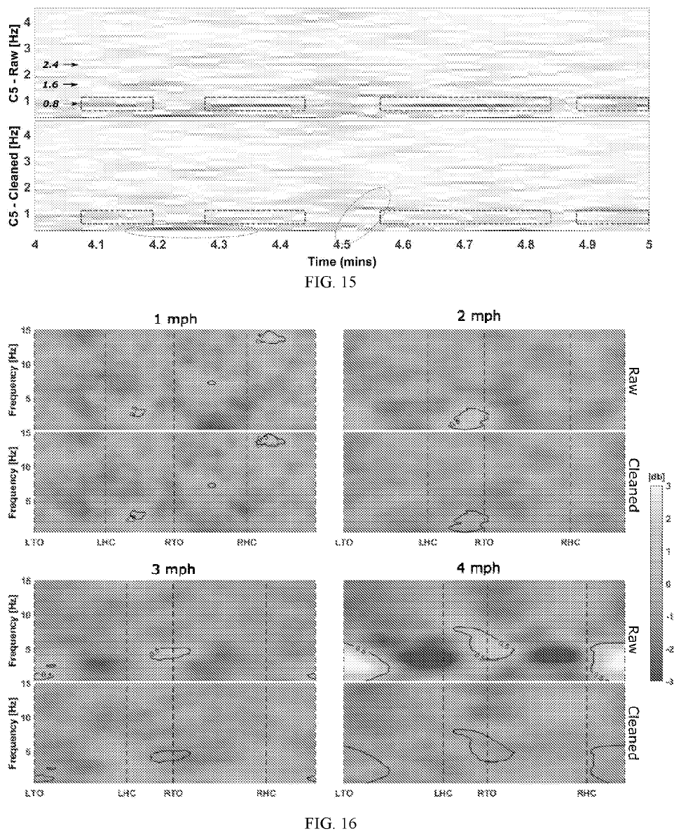

We have also tested our motion artifact cleaning framework for the same representative subject for simplicity, but for all walking speed conditions. The variability of the artifact harmonics are also prominent in all walking conditions. Plots were generated for continuous 4 minutes of treadmill walking data. Note that the slower walking speeds (especially 1-mph) have far less artifact contamination. The clean EEG spectra does not show any clear sign of remaining artifacts. It should be noted that the presence of an artifact peak or harmonics does not necessarily mean the contamination for the entire experimental duration. Rather, what we have observed is that the artifacts, especially for slower speeds, manifest themselves in a discontinuous manner, showing stronger appearance at some sections and no apparent contamination on others. FIG. 15 shows two sets of plot that summarizes the discontinuous appearances of the artifacts. FIG. 15 shows time-frequency plot of a segment of EEG data (C5 electrode) at 2 mph walking speed. Upper and lower plots show the before and after motion artifact cleaned data, respectively. 0.8 Hz higher amplitude oscillations before cleaning are marked (boxes) as a reference for after cleaning data. Example regions that remain unchanged were also marked (circles). Since the artifact dynamics are highly variable among subjects, electrode locations, a short segment of data were used to highlight the discontinuous nature of the artifacts. For the given segments, the 0.8 Hz contamination is visible before cleaning the artifacts (upper plot). Within one minute of recording, 4 segments of contamination were observed. This plot also shows the selective nature of our cleaning method as the contaminated segments were cleaned (boxes) and the rest of the time-frequency representation remain intact (circles) where no apparent contamination harmonics were expected.

The variability of artifact appearance instances and dynamics prevents us from generating group statistics for a continuous, long segment time/frequency analysis, and gauge the performance of our framework. One way of accomplishing this is investigating the event dependent nature of the artifacts to rule out the cancellation os artifactual features. For a walking task, the Event-Related Spectral Perturbations (ERSP) analysis is a good way of determining the extent of our framework. Per subject and walking speed, we have calculated the time-frequency spectrum of EEG data from all scalp locations and segmented them with respect to the gait events. We have then time warped the segments to mean gait duration and excluded the gait durations that are of above or below 3std of the mean gait duration.

FIG. 16 shows the ERSP of the C5 electrode for all walking speeds for the same subject to be able to compare the results with abovementioned outcomes. The electrode is chosen as C5 as a sample and to be in parallel with the earlier analysis. Raw (upper) and cleaned (lower) data ERSPs are shown for each speed. LTO, LHC, RTO, and RHC are gait phases and refer to left toe off, left heel contact, right toe off and right heel contact, respectively. All 4 waling speed conditions were analyzed. For each condition, the raw (upper) and cleaned (lower) data ERSPs were presented. The contour for the raw data (black) circles the above 0.5 [db] values. Same contour lines for each condition were also plotted on ERSPs of the cleaned data for comparison. The red contour plots on the bottom plots are specific to the cleaned data ERSPs.

As can be seen from the 1-mph walking condition, the raw data has gait locked events around 2.5 Hz and 14 Hz range. However, checking the entire power spectrum of the same channel (FIG. 14), 1 mph walking has strong contamination for around 0.55 Hz, which is in parallel with the actual walking speed. FIG. 14 shows power spectra of the raw and clean EEG signals for select electrodes and all experimental conditions. Some prominent harmonics were marked to show the variability of the artifacts, and selective nature of our time-domain cleaning method per condition and channel. There is no evidence that these gait locked power spectral changes for the 1-mph condition are representative of motion artifacts. Our framework keeps this information intact as can be seen from the cleaned data ERSPs. The 2-mph condition, however, shows strong gait locked activity LHC-RTO dynamic shift and the values are in the expected range for motion artifacts. Our framework marks this event as motion artifact and removes it effectively. Similarly, the 3-mph condition shows activity around the expected frequencies for motion artifacts. The activity for this walking speed becomes visible, especially for the LTO and RTO stages. In fact, ˜5 Hz activity is also visible as a harmonic peak in FIG. 14. Both the ˜1 Hz and ˜5 Hz activities were identified as motion artifacts and cleaned automatically by our framework. As expected, the gait locked activity increases with the walking speed. The 4-mph condition shows very strong activity in LTO, RTO and left dynamic shift regions. Our framework was able to identify all occurrences as artifacts and clean them.

These examples can be extended to all scalp electrode locations, subjects and speeds as we do not bound our framework, or the input data to any electrode, subject, or condition-specific variable, which are hard to measure. Instead, as justified before, the IMU data is found to be applicable for all conditions and scalp spatial locations.

Discussion: We have provided a comprehensive filtering framework for handling one of the most significant, and yet to be solved problem associated with all EEG recording paradigms, especially ones that require mobile tasks. Of course, the term mobile is used to represent any EEG recording session that results in head motion which causes motion artifacts. Our framework can be used as an offline post-processing tool for any recordings that provides a synchronized set of IMU sensor data with the EEG data. We are able to use the acceleration data of the IMU unit and used the quaternions calculated using the gyroscope and magnetometer. Although the users can calculate the quaternions via many known and very well established methods, most IMU systems provide the information as an already calculated output signal from their IMU systems, as in the APDM OPAL system used in this study. The quaternions, of course, used for compensating the gravitational acceleration constant from all axis. The resulting acceleration data is proven to be adequate as an input signal to our method. One significant advantage of our method is its real-time applicability. The gravity compensated acceleration can be measured from many commercial systems. Our method utilized this data and updated the parameters of the non-linear projection from the acceleration to each EEG channel separately, in a sample adaptive basis. This means for each sample recorded, a modified (cleaned) signal is outputted from our framework. Of course, the Volterra series expansion is not the most computationally efficient way of representing the nonlinearity. But we believe there is a good tradeoff between the performance and computational load, as we have used a 2^{nd} order representation, with 3 input signals. Our implementation (in Matlab C-MEX) on a Windows PC with dual 2.39 GHz processors is able to handle the cleaning of EEG data, from all 60 electrodes locations in real-time, with a safety margin of 6 times the real-time recording rate. For users looking for even faster processing can use the embedded coding version of our framework, or utilize a multi-threading approach, at least electrode-wise. Another alternative is to use the bilinear filter representation instead of the Volterra representation, with somewhat reduced performance, as discussed in. Another option can be the use of a reduced number of spectral target peaks. We have used all the identified spectral peaks as calculated from the IMU sensor, however one can also use every second (or nth) spectral peak, by choosing the bandwidth of the filter bank member to capture the overlapping frequencies.

For the purpose of this discussion, introducing our method as a solution to the motion artifact problem, we have focused on targeted applications and frequency ranges that most suffer from the motion artifacts. We have not done any prior processing, except for the ocular artifacts, that cleans muscle artifact, electrode pops, and missing samples. Rather our implementation is very specific to the motion artifacts. Using our method for ocular artifacts, as we have done here, we have a unified framework for real-time filtering of two of the major EEG contaminants. We have limited our efforts to the 0.3-15 Hz range as above the 15 Hz, the muscle artifacts are expected, which is beyond our target implementation. To represent our method's performance, we had to limit our efforts to a frequency range that ensures a specific contamination type, and targeted implementation for the motion artifact problem. We have, however already used our framework for beyond 15 Hz, and we believe there is no limitation of an effective cleaning of motion artifacts for the higher frequency ranges.

Additional uses of the method base: This the H^{∞} robust adaptive filter cleaning methods are also applicable to other physiological or non-physioligical artifacts, that contaminate neural signal measurements in general. Sample usages to other applications of either the original H^{∞}/TV formulation used for ocular artifact cleaning or the method presented are summarized in further discussion. The cleaning applied to simultaneous Transcranial Alternating Current Stimulation (tACS) is summarized and EEG measurement application. The stimulation on phases in the plots show only the clean data for simplicity, as the raw data amplitude is several orders of magnitude higher, and makes the comparison impossible. Stimulation off phases show both the clean and the raw data. Note that the data set is continuous with only the change in stimulation intensity and on/off times. Our filter is active at all times. As an example of recovering the underlying EEG features, the recovered eye-blink waveforms on 6 EEG channels are presented. The input to the filter is the reference stimulation waveform from the stimulation device. Examples are shown for 10 Hz, 6 Hz and 20 Hz stimulations. The cleaning of ballistocardiographic (BCG) artifacts from EEG data is also summarized, when measured simultaneously with functional Magnetic Resonance Imaging (fMRI). The figure show the before and after cleaning power spectra of a sample channel. The artifact peaks and harmonics are present in multiple frequencies on raw data, whereas clean data show no contamination, and no residual after cleaning. Here the reference signal is selected as a reference ECG sensor, or the template of artifact created from all scalp sensor data.

Our method can also be generalized when there is no reference data present. In general, EEG/fMRI gradient artifact, BCG artifacts, and many other types of artifacts are handled by building a template of the artifact via various methods, most general being the average of EEG sensor data for all channels. This average template is then subtracted from the scalp EEG sensors one by one. Although some level of cleaning is achieved using this method, often residual artifacts remain, due to amplitude variations on the artifact, and thus not following the mean template perfectly. Our robust adaptive method can receive this template as a reference input, and adapt to the amplitude changes automatically, sample-by-sample, thus leaving no residual artifacts behind, and resulting in a far better performance and precision cleaning.

Results of Removal of Other Artifacts (BCG & tACS): Subjects and Setup: All subjects provided informed consent before participating. For all experiments, Brain Products (GmbH) wet electrode systems (64 channel) have been used. For the ocular (100 Hz), BCG (5000 Hz), and motion (1000 Hz) artifact removal paradigms, 4 channels were allocated for capturing bipolar eye blink and eye-movement activity (EOG). For the motion artifact removal paradigm, subjects were also equipped with 1 forehead Inertial Measurement Unit (IMU) to capture the overall head dynamics. Finally, for the tACS artifact removal paradigm (5000 Hz), 5 channels (T7, CP2, CP5, FC3, CP3) were assigned for stimulation. An intermittent, 6 Hz stimulation frequency was used.

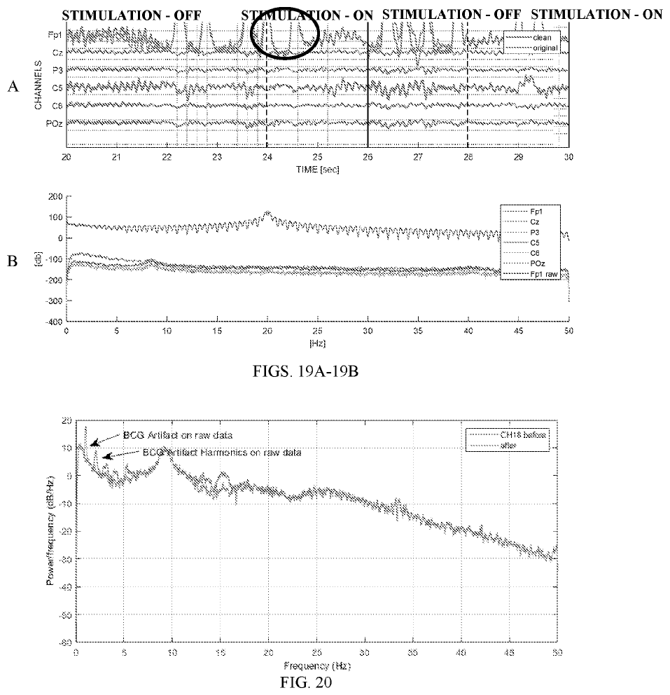

Sample Adaptive Filter for Additional Artifact Types: FIG. 20 shows cleaning of BCG artifacts from a select EEG channel. Before and after PSDs were shown. All fundamental and harmonic contaminations were cleaned. Analysis was conducted in accordance with the framework discussed above. The remaining types of artifact cleaning methods addressed also utilize the abovementioned core functionality and adaptability to time domain changes provided by the H^{∞} filter with time-varying weight assumption. Sample applications are discussed in this section, introducing also the artifact specific differences in processing pipelines.

BCG/EEG artifacts: Methods that utilize the average template of the artifacts for subtracting it from the measurements are very common in neural signal denoising. The biggest problem for these methods is to eliminate the residual artifacts after cleaning due to the time domain amplitude variations in the signal, and thus (short duration) mismatch of the template and the artifacts. We have utilized our adaptation scheme to accommodate these variations for the BCG signals. Instead of using the common approach of handling residuals via an ANC method after standard template subtraction, we have used our H^{∞} method as the subtraction step utilizing also the adaptation to amplitude variations, simultaneously. After cleaning the EEG of gradient artifact using standard tools, we cleaned the EEG data from ocular artifacts using our abovementioned method. Then an ECG average was calculated using the contaminated channels to serve as a template. This template, without further processing, was used as a reference signal to our H^{∞} framework. FIG. 14 shows the before and after PSDs of a representative channel of EEG (Cz). The ˜1 Hz contamination and its harmonics, were cleaned efficiently, where the rest of the spectrum remain unchanged.

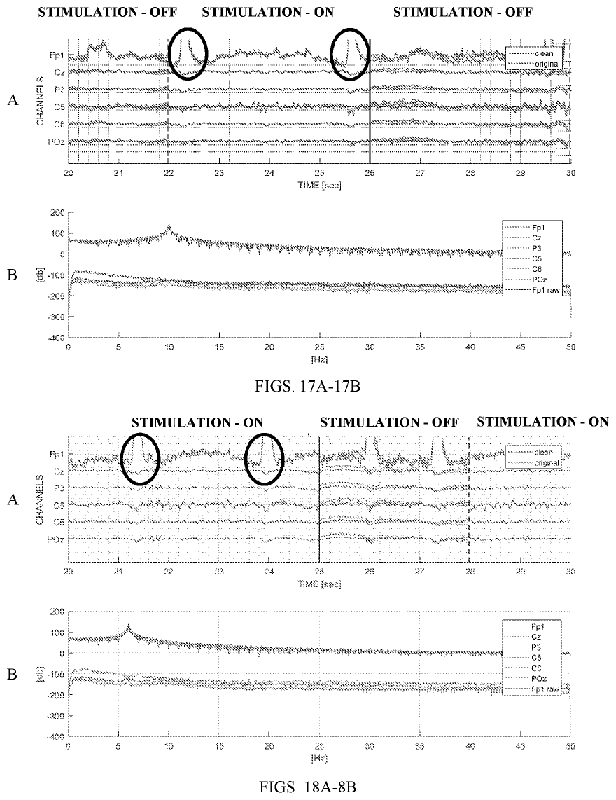

tACS/EEG artifacts: FIGS. 17A-17B show simultaneous tACS/EEG stimulation artifact cleaning for 10 Hz discontinuous stimulation. Note the recovered ocular data on frontal channels (marked with black ellipses). FIGS. 18A-18B show simultaneous tACS/EEG stimulation artifact cleaning for 6 Hz discontinuous stimulation. Note the recovered ocular data on frontal channels (marked with black ellipses). FIGS. 19A-19B simultaneous tACS/EEG stimulation artifact cleaning for 20 Hz discontinuous stimulation. Note the recovered ocular data on frontal channels (marked with black ellipses).

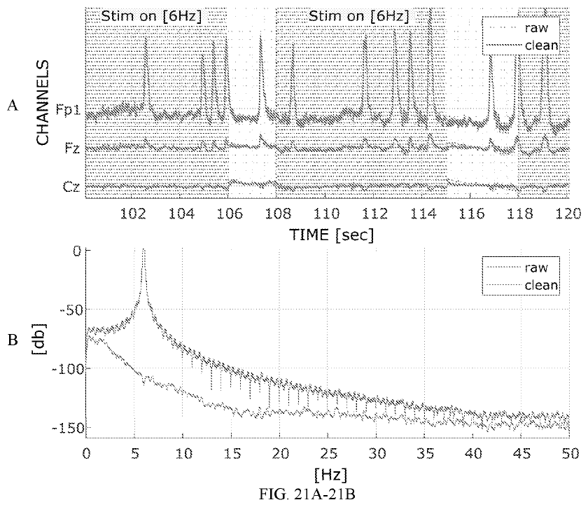

The tACS stimulation artifacts manifest themselves as very high amplitude contaminants (˜10⁴ times the expected EEG amplitude). Similar to the BCG removal method described above, we have generated a template of the contamination to be used as a reference signal for our method. To accomplish this, first the nonlinear drifts in the data set were removed using a 5^{th} order polynomial (corresponding to lower than 0.1 Hz oscillations). EEG data amplitudes are then normalized to the [−1 1] range, while saving the scaling coefficients (calculated using a clean segment) for later use. Then each EEG channel data were smoothed using a Savitzky-Golay filter (order 7 and frame length of 301). This smoothing is intended to eliminate actual neural signals oscillations superimposed onto the high amplitude artifacts. We then implemented a Hilbert transform and derived the instantaneous phase and amplitude of the signal and generated a synthetic data set that has amplitude of 1 and phase identical to the values derived from the Hilbert transform. At this point, the synthetic data has no amplitude modulation information, but the phase information matches that of the average artifacts. Using this phase matched reference signal, we have implemented our H^{∞} method to clean the EEG and scaled the output back to original levels using the saved amplitude scaling coefficients. FIG. 20 (top) shows the before and after cleaning of the data for select channels. The raw data were not scaled for a better visual representation, and accommodate large amplitude differences. It should be noted that our filter is active for the full duration of the dataset, processing the data in a sample-adaptive fashion, including when the artifacts are not present. As seen in the figure, the eyeblinks were recovered after cleaning and are visible on both stimulation-on and stimulation-off sections of the dataset. Note that the eye blinks also have components in 6 Hz (stimulation frequency). Our method was successful in selectively removing only the stimulation artifacts and keeping the reminder of the underlying data intact. The PSD of the clean data (FIG. 21B—bottom) also shows no residual contamination, or harmonics. FIGS. 1A-21B show a segment of raw and clean EEG data for the intermittent tACS stimulation paradigm (A), and the corresponding PSDs before and after cleaning (B).

Conclusion: Our novel processing framework, and its sample adaptive formulation can be applied to multiple types of artifactual neural measurements. Our implementation is already real-time compatible for the ocular and motion artifacts. For the BCG artifacts, instead of using the template as a reference channel, a dedicated ECG channel can be used, which makes the practical implementation real-time compatible. For the tACS/EEG methodology, the signal could be buffered to include at least two artifactual oscillations to generate an initial template, and this template can be updated using a circular buffer implementation, which allows for real-time use. Our method can also be used as an offline tool for post-processing, allowing for the recovery of the valuable underlying information content. Considering the statistical similarities, our method can also be applied to a multitude of neural measurement paradigms, including fNIRS and MEG, and can also be used for other types of artifacts, including EMG and gradient artifacts.

Embodiments described herein are included to demonstrate particular aspects of the present disclosure. It should be appreciated by those of skill in the art that the embodiments described herein merely represent exemplary embodiments of the disclosure. Those of ordinary skill in the art should, in light of the present disclosure, appreciate that many changes can be made in the specific embodiments described, including various combinations of the different elements, components, steps, features, or the like of the embodiments described, and still obtain a like or similar result without departing from the spirit and scope of the present disclosure. From the foregoing description, one of ordinary skill in the art can easily ascertain the essential characteristics of this disclosure, and without departing from the spirit and scope thereof, can make various changes and modifications to adapt the disclosure to various usages and conditions. The embodiments described hereinabove are meant to be illustrative only and should not be taken as limiting of the scope of the disclosure.

What is claimed is:

1. A method removing artifacts from neural signals, the method comprising the steps of:
receiving electroencephalography (EEG) data from an EEG system;
providing the EEG data to a unified artifact removal framework for cleaning artifacts, wherein the EEG data is provided to a first cleaning framework;
providing a first reference signal to the first cleaning framework, wherein the first reference signal is utilized to clean first artifacts from the EEG data;
outputting the EEG data from the first cleaning framework to a second cleaning framework;
providing a second reference signal to the second cleaning framework, wherein the second reference signal is utilized to clean second artifacts from the EEG data; and
outputting fully cleaned EEG data from the unified artifact removal framework.

2. The method of claim 1, wherein the first or second cleaning frameworks apply an H^{∞} adaptation rule to the first or second reference signals, and a negative of an output after application the H^{∞} adaptation rule is combined with input EEG data to clean the first or second artifacts.

3. The method of claim 1, wherein the first cleaning framework is an ocular cleaning framework utilized to clean ocular artifacts from the EEG data, the first reference signal is electrooculography (EOG) data,
the ocular cleaning framework applies an H^{∞} adaptation rule to the EOG data, and a negative of an output after application the H^{∞} adaptation rule is combined with the EEG data to output ocular cleaned EEG data.

4. The method of claim 1, wherein the first cleaning framework is a cascade filtering framework for cleaning motion artifacts, inertial measurement unit (IMU) data gathered from an IMU positioned on a subject's head is the second reference signal,
the cascade filtering framework applies a Volterra expansion to the IMU data and applies a H^{∞} adaptation rule to an output IMU data subjected to the Volterra expansion, and a negative of an output after application the H^{∞} adaptation rule is combined with incoming EEG data to output motion artifact cleaned EEG data.

5. The method of claim 4, wherein the cascade filtering framework comprises two or more stages, where the steps of applying the Volterra expansion, applying the H^{∞} adaptation rule, and combining the negative of the output with the incoming EEG data are repeated in each stage, and output EEG data is fed to a subsequent stage.

6. The method of claim 1, wherein the unified artifact removal framework further comprises a ballistocardiography (BCG) framework for cleaning BCG artifacts, the BCG framework using an electrocardiography (ECG) average as a template for a reference, and the BCG framework performs the steps of
applying an H^{∞} adaptation rule to the reference; and
combining an output after application the H^{∞} adaptation rule with incoming EEG data to clean the BCG artifacts.

7. The method of claim 1, wherein the unified artifact removal framework further comprises a transcranial alternating current stimulation (tACS) framework for cleaning tACS artifacts, using tACS artifact data as a reference, and the tACS framework performs the steps of
applying an H^{∞} adaptation rule to the reference; and
combining an output after application the H^{∞} adaptation rule with incoming EEG data to clean the ECG artifacts.

8. The method of claim 2, wherein the H^{∞} adaptation rule is:
$${\hat{w}}_{i + 1} = + \frac{{P}_{i} {r}_{i}}{1 + {r}_{i}^{T} {P}_{i} {r}_{i}} {y}_{i} for which , \\ {y}_{i} = {s}_{i} - {r}_{i}^{T} , and {P}_{i}^{- 1} = {\tilde{P}}_{i}^{- 1} - {γ}^{- 2} {r}_{i} {r}_{i}^{T} where$$
$${\tilde{P}}_{i + 1} = {[ {{\tilde{P}}_{l}}^{- 1} + ( 1 - {γ}^{- 2} ) {r}_{i} {r}_{i}^{T} ]}^{- 1} + qI$$
where ŵᵢ represent the estimated weight vector of reference values, rᵢ the reference vector at sample i, sᵢ represents the EEG data, and \tilde{P}ᵢ is the noise covariance matrix initialized with \tilde{P}₀=μl where μ is a constant, γ determines bounds on the energy-to-energy gain from a disturbance to estimation error, q reflects a priori information of how rapidly weight varies in time.

9. The method of claim 1, wherein the fully cleaned EEG data is utilized to control a prosthetic or exoskeleton.

10. A system for removing artifacts from neural signal comprising:
an electroencephalography (EEG) system comprising a plurality of electrodes, wherein the plurality of electrodes are capable of gathering EEG data;
a unified artifact removal framework for cleaning artifacts receiving the EEG data, wherein the unified artifact removal framework comprises one or more artifact removal frameworks, and each of the one or more artifact removal frameworks comprise
a H^{∞} module receiving a reference utilized to clean artifacts from the EEG data, and
a combiner combining an output of the H^{∞} module with the EEG data to clean the artifacts from the EEG data;
an output outputting fully cleaned EEG data from the unified artifact removal framework; and
a processor controlling operation of the unified artifact removal framework.

11. The system of claim 10, wherein the H^{∞} module applies an H^{∞} adaptation rule to the reference, and a negative of the output of the H^{∞} module is combined with the EEG data.

12. The system of claim 10, wherein a subset of the plurality of electrodes are ocular electrodes for measuring an ocular reference that is electrooculography (EOG) data,
the unified artifact removal framework comprises an ocular cleaning framework utilized to clean ocular artifacts from the EEG data, the ocular cleaning framework comprises
an ocular H^{∞} module applying an H^{∞} adaptation rule to the EOG data, and
a combiner combining an output of the ocular H^{∞} module with the EEG data to clean ocular artifacts from the EEG data.

13. The system of claim 10, wherein the unified artifact removal framework comprises a cascade filtering framework for cleaning motion artifacts,
the EEG system further comprises an inertial measurement unit (IMU) positioned on a subject's head gathering IMU data utilizes as an IMU reference,
the cascade filtering framework comprising
a Volterra expansion module for applying a Volterra expansion to the IMU data,
an IMU H^{∞} module applying an H^{∞} adaptation rule to an output of the Volterra expansion module, and
a combiner combining an output of the IMU H^{∞} module with the EEG data to clean the motion artifacts from the EEG data.

14. The system of claim 13, wherein the cascade filtering framework comprises two or more stages, where each stage comprises a Volterra expansion module, an IMU H^{∞} module, and a combiner.

15. The system of claim 10, wherein the unified artifact removal framework further comprises a ballistocardiography (BCG) framework for cleaning BCG artifacts, the BCG framework using an electrocardiography (ECG) average as a template for a reference, and the BCG framework comprises
an BCG H^{∞} module applying an H^{∞} adaptation rule to the ECG average, and
a combiner combining an output of the BCG H^{∞} module with the EEG data to clean the BCG artifacts from the EEG data.

16. The system of claim 10, wherein the unified artifact removal framework further comprises a transcranial alternating current stimulation (tACS) framework for cleaning tACS artifacts, using tACS data as a reference, and the tACS framework comprises
a tACS H^{∞} module applying an H^{∞} adaptation rule to the tACS data, and
a combiner combining an output of the tACS H^{∞} module with the tACS data to clean the tACS artifacts from the EEG data.

17. The system of claim 11, wherein the H^{∞} adaptation rule is:
$${\hat{w}}_{i + 1} = + \frac{{P}_{i} {r}_{i}}{1 + {r}_{i}^{T} {P}_{i} {r}_{i}} {y}_{i} for which , \\ {y}_{i} = {s}_{i} - {r}_{i}^{T} , and {P}_{i}^{- 1} = {\tilde{P}}_{i}^{- 1} - {γ}^{- 2} {r}_{i} {r}_{i}^{T} where$$
$${\tilde{P}}_{i + 1} = {[ {{\tilde{P}}_{l}}^{- 1} + ( 1 - {γ}^{- 2} ) {r}_{i} {r}_{i}^{T} ]}^{- 1} + qI$$
where ŵᵢ represent the estimated weight vector of reference values, rᵢ the reference vector at sample i, sᵢ represents the EEG data, and \tilde{P}ᵢ is the noise covariance matrix initialized with \tilde{P}₀=μl where μ is a constant, γ determines bounds on the energy-to-energy gain from a disturbance to estimation error, q reflects a priori information of how rapidly weight varies in time.

18. The method of claim 10, wherein the fully cleaned EEG data is utilized to control a prosthetic or exoskeleton.

19. A method removing artifacts from neural signals, the method comprising the steps of:
receiving electroencephalography (EEG) data from an EEG system;
providing the EEG data to a unified artifact removal framework for cleaning artifacts,
wherein the unified artifact removal framework comprises an ocular cleaning framework, and the EEG data is provided to the ocular cleaning framework;
providing a first reference to the ocular cleaning framework, wherein the first reference signal is utilized to clean ocular artifacts from the EEG data;
wherein further the unified artifact removal framework comprises a cascade filtering framework, and output EEG data from the ocular cleaning framework is provided to the cascade filtering framework;
providing a second reference to the cascade filtering framework, wherein the second reference is utilized to clean motion artifacts from the second input; and
outputting fully cleaned EEG data from the unified artifact removal framework after cleaning the ocular artifacts and the motion artifacts.

20. The method of claim 19, wherein the fully cleaned EEG data is utilized to control a prosthetic or exoskeleton.
